(19) United States
(12) Patent Application Publication
(10) Pub. No.: US 2023/0365994 A1
(43) Pub. Date: Nov. 16, 2023

(54) HSV VECTORS WITH ENHANCED REPLICATION IN CANCER CELLS

(71) Applicant: VIROGIN BIOTECH CANADA LTD, Richmond (CA)

(72) Inventors: William Jia, Vancouver (CA); Dmitry Chouljenko, Vancouver (CA); Xuexian Bu, Surrey (CA); Guoyu Liu, Richmond (CA)

(21) Appl. No.: 18/305,366

(22) Filed: Apr. 23, 2023

Related U.S. Application Data
(63) Continuation of application No. 16/095,077, filed on Oct. 19, 2018, now abandoned.
(60) Provisional application No. 62/329,877, filed on Apr. 29, 2016.

(51) Int. Cl. C12N 15/86; C12N 7/00; C07K 14/035; A61P 35/00; A61K 35/768; C07K 14/54

(52) U.S. Cl. CPC C12N 15/86; C12N 7/00; C07K 14/035; A61P 35/00; A61K 35/768; C07K 14/5434; C07K 14/5443; C12N 2710/16621; C12N 2830/002; C12N 2710/16633; C12N 2710/16643; C12N 2830/34

(57) ABSTRACT

An oncolytic HSV vector comprising an NF-κB response element or an Oct-3/4-SOX2 response element in a regulatory region of a viral gene that affects viral replication efficiency.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### FIELD OF THE INVENTION

This patent application relates generally to enhancing replication of HSV in cancer cells.

### REFERENCE TO SEQUENCE LISTING, TABLE OR COMPUTER PROGRAM

The contents of the electronic sequence listing (VIRO_402 USC1_seqlist_ST26.text.xml; Size: 60.0 KB and Date of Creation: Apr. 23, 2023) is herein incorporated by reference in its entirety.

### BACKGROUND OF THE INVENTION

Oncolytic viruses (OVs) have been a therapeutic arsenal to specifically destroy cancer cells through oncolysis, which is a killing mechanism characterized by cancer cell lysis through the course of virus lytic replication. In addition to the direct cell killing by the virus. Among the various OVs, herpes simplex virus type 1 (“HSV-1”) based OVs are the farthest advanced, e.g., a herpes virus-based OV (T-Vec) has been approved by the U.S. FDA for the treatment of melanoma. Representative examples of HSV vectors include those described in U.S. Pat. Nos. 7,223,593, 7,537,924, 7,063,835, 7,063,851, 7,118,755, 8,277,818, and 8,680,068.

The present invention overcomes shortcomings of current commercial oncolytic viruses, and further provides additional unexpected benefits.

### SUMMARY

Briefly stated, the present invention provides HSV vectors with enhanced replication in cancer cells. Within one embodiment HSV vectors are provided comprising an NF-κB response element in a regulatory region of a viral gene that affects viral replication efficiency. Other claims are directed to an HSV vector comprising an Oct3/4-SOX2 response element in a regulatory region of viral gene that affects viral replication efficiency. The viral genes that may have one or both of these response elements include US11 and genes that encode ICP4, ICP27 and ICP8.

Other claims are directed to a vector, wherein the NF-κB response element comprises from 1-15 tandem sequences of GGGAATTTCC (SEQ ID NO: 1) or variants thereof, wherein the variant has at least one nucleotide difference from this sequence. The tandem sequences may be identical or a mix of identical and different sequences or all different sequences. Other claims are directed to a vector, wherein the OCT3/4-SOX2 response element comprises CTACAGAGGTGCATATTAACAGAGCTTTTGTCCTGGAGA (SEQ ID NO: 2) or a variant thereof, wherein the variant has at least 90% identical nucleotides.

In other claims, the HSV vector may further comprise a sequence encoding a therapeutic substance for cancer treatment. The therapeutic substance may be IL12, IL15, OX40L, PDL-1 blocker or a PD-1 blocker. One example of suitable blocker is described in U.S. patent application Ser. No. 15/374,893, filed on Dec. 9, 2016, hereby incorporated in its entirety.

Other claims are directed to a method of treating cancer, comprising administering an HSV vector as described herein to a patient with cancer or suspected of having cancer. In other claims, a method of treating cancer stem cells and treatment-resistant cancer is provided, comprising administering an HSV vector as described herein to a patient with a treatment resistant cancer or a cancer having cancer stem cells. In other claims, the cancers are colon cancer, lung cancer, breast cancer, prostate cancer, brain cancer, or bladder cancer.

The SUMMARY has been provided to introduce certain concepts in a simplified form that are further described in detail below in the DETAILED DESCRIPTION OF THE INVENTION. Except where otherwise expressly stated, this SUMMARY is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIGS. 1A and 1B are schematics of exemplary oHSV vectors.

FIG. 2 is a graph showing enhanced viral production of an exemplary oHSV-1 vector carrying a NF-κB response element in the regulatory region of ICP4 in the presence of TNFα.

FIGS. 3A and 3B present the schematic and sequence (SEQ ID NO: 3) of OS-ICP27.

FIGS. 4A and 4B present the schematic and sequence (SEQ ID NO: 4) of NO-ICP27-145.

FIGS. 5A and 5B present the schematic and sequence (SEQ ID NO: 5) of NO-ICP27-99.

FIG. 6 shows a schematic of the modified ICP34.5 region (SEQ ID NO: 6) for virus hVG161.

FIG. 7 shows a schematic of a modified UL54 promoter region (SEQ ID NO: 7) for virus hVG161.

FIG. 8 shows a schematic of hVG161.viral genome with an insertion of a PD-L1 blocker (SEQ ID NO: 8).

FIG. 9 shows a schematic of hVG161 modified TR region (SEQ ID NO: 9).

FIGS. 10A, 10B, 10C, 10D, 10E, 10F, 10G, 10H, 10I, 10J and 10K show assay results for a virus comprising mutations in ICP27 promoter/regulatory region.

FIGS. 11A, 11B, and 11C show ELISA and Western blot data for IL-12 expression following hVG161 infection of cells.

FIGS. 12A, 12B, and 12C show ELISA and Western blot data for IL-15 expression following hVG161 infection of cells.

FIGS. 13A, 13B, and 13C show ELISA and Western blot data for IgG4 expression following hVG161 infection of cells.

FIGS. 14A, 14B, 14C, 14D, and 14E show results of cell infection with VG161-PLBh and VG161-15h.

FIGS. 15A, 15B, 15C, and 15D show results of in vitro assays for various constructs. FIG. 15A-15B show results of cell transfection with IL-TF-Fc plasmid carrying IL-12, IL-15, and PD-L1 blocker. FIGS. 15C-15D show results of cell infection with a variety of mutant viruses including hVG161.

FIGS. 16A, 16B, 16C, 16D, and 16E show results of cell viability assays for hVG161 and HSV-345 on human tumor cell lines and Vero cell line.

FIGS. 17A, 17B, 17C, 17D, 17E, 17F, 17G, 17H, 17I, and 17J show results of in vitro assays for various constructs. FIGS. 17A-17E show results of cell viability assays for mVG161 and HSV-345 on mouse tumor cell lines and Vero cell line; FIGS. 17F-17J show the characterization of transgene expression following mVG161 or VG001 infection of CT26 mouse tumor cells.

FIGS. 18A, 18B, 18C, 18D, and 18E show results of in vitro characterization of transgene expression following hVG161 or VG001 infection of various cell lines.

FIGS. 19A, 19B, 19C, 19D, 19E, 19F, and 19G show results of assays to evaluate the ability of hVG161 to kill a variety of human cancer cells in vitro.

FIGS. 20A, 20B, 20C, 20D, 20E, 20F, and 20G show results of in vivo assays for mVG161 and hVG161 constructs.

FIGS. 21A, 21B, and 21C show growth curves for different viruses on three different human cell lines.

FIGS. 22A, 22B, 22C, and 22D show growth curves of mVG161 and HSV-345 on mouse tumor cell lines and Vero cell line.

FIGS. 23A, 23B, 23C, 23D, and 23E show growth curves of hVG161 and HSV-345 on human tumor cell lines and Vero cell line.

FIGS. 24A, 24B, 24C, and 24D show growth curves of NO-ICP27 and HSV-345 on various cell lines.

### DETAILED DESCRIPTION OF THE INVENTION

The present invention may be understood more readily by reference to the following detailed description of preferred embodiments of the invention and the Examples included herein. Briefly stated, the present disclosure provides compositions and methods for treating cancer using HSV vectors that comprise sequences that are responsive to NF-κB or Oct-3/4-SOX2 or both transcriptional activators.

Within certain embodiments of the invention, the present disclosure provides methods and compositions, to cancer cells, typically in vivo to a subject in need of cancer treatment. The term “cancer,” as used herein, refers to a cancer of any kind and origin, including treatment resistant cancers (e.g., resistant to conventional radiation and/or chemotherapy); cancer stem cells, tumor-forming cells, blood cancers, and transformed cells. The term “cancer cell,” as used herein, includes cancer or tumor-forming cells, transformed cells, or a cell that is susceptible to becoming a cancer or tumor-forming cell. Representative forms of cancer include carcinomas, sarcomas, myelomas, leukemia's, lymphomas, and mixed types of the above. Further examples include, but are not limited to those discussed in more detail below.

Some cancer cells, especially cancer stem cells, are relatively resistant to oncolytic HSV-1 virus induced oncolysis. To increase the therapeutic efficacy of oHSV-1, the viral genes that regulate viral replication can be modified in their regulatory region to insert an NFkB response element or an Oct3/4-SOX2 response element or both to make the gene expression particularly responsive to activation of NFkB in infected cells. Since NFkB pathway is often upregulated in cancer cells and cancer stem cells, this modification will enhance viral production and oncolytic activity of oHSV for more effective cell killing.

### A. NF-κB and Oct-3/4-SOX2 Response Elements

Oct-3/4-SOX2 complex (also designated Oct-3, Oct4 and Pou5f1) and NF-κB are expressed in many different tumors. Oct4 and SOX2 are transcriptional factor expressed in stem cells. The two proteins form a complex to bind to the regulatory regions of genes involved in maintaining stem cells and pluripotent cells. In cancer stem cells it maintains a “sternness” state. Cancer stem cells have been identified in a variety of solid malignancies. They are a small population of tumor cells with stem cell characteristics, which are a likely cause of relapse in cancer patients. NF-κB (Nuclear factor-kappaB) is a family of transcription factors. In essentially all unstimulated nucleated cells, NFκB complexes are bound to an inhibitor of NFκB (IκB) proteins. When released from the inhibitor, NF-κB translocates into the nucleus and targets a sequence in enhancer elements, activating or increasing gene transcription. NF-κB is activated in many cancer cell types, including hepatocellular carcinoma, lymphomas, leukemias, colon cancer, lung cancer, breast cancer, prostate cancer, brain cancer, and bladder cancer. It is also activated in cancer stem cells.

Response elements for NFκB includes the decameric binding sequence. The most common sequence is GGGAATTTCC (SEQ ID NO: 1), although variants can be targets as well. In general, a variant will have one or two differences to the most common sequence.

Some known NFκB binding sequence variants include:

TABLE 1

| SEQUENCE | SEQ ID NO. |
|---|---|
| GGGGATTTCC | 10 |
| GGGAGATTCC | 11 |
| GGGAATCTCC | 12 |
| GGGAGATTCC | 13 |
| GGGGATTCCCC | 14 |
| GGGGAATCCC | 15 |
| GGGAATCCCC | 16 |
| GGGGAAGGCC | 17 |
| GGGAATTTCC | 18 |
| GGGAAATTCC | 19 |
| GGGAACTACC | 20 |
| GGGATTTTCC | 21 |
| GGGATTTCAC | 22 |
| GGGGCTTTCC | 23 |
| GGGAAAGCCC | 24 |
| GGGAATTCAC | 25 |
| GGGGGCTTCC | 26 |
| GGGGCTTCCC | 27 |
| GGGAAGCCCC | 28 |
| GGGGAAGCCC | 29 |
| GGGAAATCCC | 30 |
| GGGACTTTCC | 31 |
| GGGGCTTTCC | 32 |
| GGGGAATCCC | 33 |
| GGGATTCCCC | 34 |

The NF-κB response element comprises at least one of the binding sequences and may comprise from 2 up to 15 binding sequences in tandem array. When more than one sequence is present, the more than one sequence may be identical or different. For example, a response element comprising five binding sequences may have all five sequences identical, or four identical and one different, or three identical and two different, or two identical and three different, or five different sequences. As well, the tandem response elements may have nucleotides in between the elements. There may be from one to about ten additional nucleotides.

Oct-3/4 binds an octameric sequence ATTTGCAT. Sox2 binds to an octameric sequence CTTTTGTC. It has been found that Oct4/Sox2 heterodimer binds to CTACAGAGGTGCATATTAACAGAGCTTTTGTCC-TGGAGA [SEQ ID No. 2] or variants of the sequence. (Wang et al. J Biol Chem 282: 12822, 2007). Variants will generally be at least 90% identical to this sequence, or at least 95% identical, or at least 98% identical.

The Oct-3/4-Sox2 response element comprises at least one of the binding sequences and may comprise from 2 up to 15 binding sequences in tandem array. When more than one sequence is present, the more than one sequence may be identical or different. For example, a response element comprising five binding sequences may have all five sequences identical, or four identical and one different, or three identical and two different, or two identical and three different, or five different sequences. As well, the tandem response elements may have nucleotides in between the elements. There may be from one to about ten additional nucleotides.

Some possible variants of the sequence for binding the Oct4/Sox2 complex can be found in MOLECULAR AND CELLULAR BIOLOGY (July 2005, p. 6031-6046), and include:

SEQ ID NO. 35
CTTTGTTATGCATCT
SEQ ID NO. 36
CATTGTGATGCATAT
SEQ ID NO. 37
CATTGTAATGCAAAA
SEQ ID NO. 38
CATTGTTATGCTAGT
SEQ ID NO. 39
CATTGTTATGATAAA
SEQ ID NO. 40
CTTTGTTTGGATGCTAAT.

The vectors may contain a mixture of NF-κB and Oct-3/4-Sox 2 response elements. When each is present, generally the response elements will be in tandem array, such as seen in the constructs of Example 2. The order of the response elements may be with either element in the 5′ position. It is also contemplated that there can be more than one of NF-κB or more than one of Oct-3/4-Sox 2 response elements or more than one of each response elements. In the case where there are multiple of one or more of the elements, they may be in any order. As well, there may be additional nucleotides between the response elements.

### B. HSV Vector Constructs

An oncolytic virus is a virus that will lyse cancer cells (oncolysis), preferably in a selective manner. Viruses that selectively replicate in dividing cells over non-dividing cells are often oncolytic.

Herpes Simplex Virus (HSV) 1 and 2 are members of the Herpesviridae family, which infect humans. The HSV genome contains two unique regions, which are designated unique long (UL) and unique short (US) region. Each of these regions is flanked by a pair of inverted terminal repeat sequences. There are about 75 known open reading frames. The viral genome has been engineered to develop oncolytic viruses for use in e.g. cancer therapy. Tumor-selective replication of HSV is conferred by mutation of the HSV ICP34.5 (also called γ34.5) gene. HSV contains two copies of ICP34.5. Mutants inactivating one or both copies of the ICP34.5 gene are known to lack neurovirulence, i.e. be avirulent/non-neurovirulent and be oncolytic.

Suitable oncolytic HSV may be derived from either HSV-1 or HSV-2, including any laboratory strain or clinical isolate. In some embodiments, the oHSV may be or may be derived from one of laboratory strains HSV-1 strain 17, HSV-1 strain F, or HSV-2 strain HG52. In other embodiments, it may be of or derived from non-laboratory strain JS-1. Other suitable HSV-1 viruses are in the table 2 below.

TABLE 2

| Virus | Name | references |
|---|---|---|
| HSV-1 | HrrR3 | [1] |
| | G2O7 | [2, 3] |
| | G47Delta | [4] |
| | HSV 1716 | [5, 6] |
| | HF10 | [7] |
| | NV1020 | [8] |
| | T-VEC | [9] |
| | J100 | [10] |
| | M002 | [11] |
| | NV1042 | [12] |
| | G2O7-IL2 | [13] |
| | rQNestin34.5 | [14] |
| | G47Δ-mIL-18 | [15] |

In some embodiments, the oHSV has one or both of the γ34.5 genes are modified such that it is incapable of expressing a functional ICP34.5 protein. The genes may be modified by mutation of one or more nucleotides, insertions, deletions, substitutions, etc. The alteration may be in the coding sequence, non-coding sequence (e.g., promoter) or both. In some embodiments, both copies of the γ34.5 genes are mutated.

The oHSV may have additional mutations, which may include disabling mutations e.g., deletions, substitutions, insertions), which may affect the virulence of the virus or its ability to replicate. For example, mutations may be made in any one or more of ICP6, ICPO, ICP4, ICP27, ICP47, ICP 24, ICP56. Preferably, a mutation in one of these genes (optionally in both copies of the gene where appropriate) leads to an inability (or reduction of the ability) of the HSV to express the corresponding functional polypeptide. In some embodiments, the promoter of a viral gene may be substituted with a promoter that is selectively active in target cells or inducible.

The oHSV may also have genes and nucleotide sequences that are non-HSV in origin. For example, a sequence that encodes a prodrug, a sequence that encodes a cytokine or other immune stimulating factor, a tumor-specific promoter, an inducible promoter, an enhancer, a sequence homologous to a host cell, among others may be in the oHSV genome. Exemplary sequences encode IL12, IL15, OX40L, PDL-1 blocker or a PD-1 blocker.

The regulatory region of viral genes encoding US11, ICP4, ICP27, and ICP8 may be modified to comprise a response element for NF-κB or Oct-3/4-SOX2 or both NF-κB and Oct-3/4-SOX2. (ICP4 is encoded by RS1; US11 is encoded by US11; ICP27 is encoded by UL54; ICP8 is encoded by UL29. The HSV-1 vector can have one viral gene that contains one or both response elements. Alternatively, the vector can have more than one viral gene that contains one or both response elements. In general, the response element will be in the viral gene enhancer region. The response element can replace or be in addition to the viral enhancer. The viral promoter may be replaced, generally with a tumor-specific promoter, such as survivin promoter. Other tumor-specific promoters are known in the art. Other gene elements may be modified as well. For example, the 5′ UTR of the viral gene may be replaced with an exogenous UTR.

### C. Therapeutic Compositions

Therapeutic compositions are provided that may be used to prevent, treat, or ameliorate the effects of a cancer. More particularly, some therapeutic compositions comprise an oncolytic virus as described herein. Within preferred embodiments, the therapeutic composition can comprise an oncolytic virus as described herein.

In certain embodiments, the compositions will further comprise a pharmaceutically acceptable carrier. The phrase “pharmaceutically acceptable carrier” is meant to encompass any carrier, diluent or excipient that does not interfere with the effectiveness of the biological activity of the oncolytic virus and that is not toxic to the subject to whom it is administered (see generally Remington: The Science and Practice of Pharmacy, Lippincott Williams & Wilkins; 21st ed. (May 1, 2005 and in The United States PharmacopE1A: The National Formulary (USP 40—NF 35 and Supplements).

In the case of an oncolytic virus as described herein, non-limiting examples of suitable pharmaceutical carriers include phosphate buffered saline solutions, water, emulsions (such as oil/water emulsions), various types of wetting agents, sterile solutions, and others. Additional pharmaceutically acceptable carriers include gels, bioadsorbable matrix materials, implantation elements containing the oncolytic virus, or any other suitable vehicle, delivery or dispensing means or material(s). Such carriers can be formulated by conventional methods and can be administered to the subject at an effective dose. Additional pharmaceutically acceptable excipients include, but are not limited to, water, saline, polyethyleneglycol, hyaluronic acid and ethanol. Pharmaceutically acceptable salts can also be included therein, e.g., mineral acid salts (such as hydrochlorides, hydrobromides, phosphates, sulfates, and the like) and the salts of organic acids (such as acetates, propionates, malonates, benzoates, and the like). Such pharmaceutically acceptable (pharmaceutical-grade) carriers, diluents and excipients that may be used to deliver the oHSV to a target cancer cell will preferably not induce an immune response in the individual (subject) receiving the composition (and will preferably be administered without undue toxicity).

The compositions provided herein can be provided at a variety of concentrations. For example, dosages of oncolytic virus can be provided which ranges from about 106 to about 109 pfu. Within further embodiments, the dosage form can range from about 106 to about 108 pfu/ml, with up to 4 mls being injected into a patient with large lesions (e.g., >5 cm) and smaller amounts (e.g., up to 0.1 mls) in patients with small lesions (e.g., <0.5 cm) every 2-3 weeks, of treatment.

Within certain embodiments of the invention, lower dosages than standard may be utilized. Hence, within certain embodiments less than about 106 pfu/ml (with up to 4 mls being injected into a patient every 2-3 weeks) can be administered to a patient.

The compositions may be stored at a temperature conducive to stable shelf-life, and includes room temperature (about 20° C.), 4° C., −20° C., −80° C., and in liquid N2. Because compositions intended for use in vivo generally don't have preservatives, storage will generally be at colder temperatures. Compositions may be stored dry (e.g., lyophilized) or in liquid form.

### D. Administration

In addition to the compositions described herein, various methods of using such compositions to treat or ameliorate cancer are provided, comprising the step of administering an effective dose or amount of a HSV vector as described herein to a subject.

The terms “effective dose” and “effective amount” refers to amounts of the oncolytic virus that is sufficient to effect treatment of a targeted cancer, e.g., amounts that are effective to reduce a targeted tumor size or load, or otherwise hinder the growth rate of targeted tumor cells. More particularly, such terms refer to amounts of oncolytic virus that is effective, at the necessary dosages and periods of treatment, to achieve a desired result. For example, in the context of treating a cancer, an effective amount of the compositions described herein is an amount that induces remission, reduces tumor burden, and/or prevents tumor spread or growth of the cancer. Effective amounts may vary according to factors such as the subject's disease state, age, gender, and weight, as well as the pharmaceutical formulation, the route of administration, and the like, but can nevertheless be routinely determined by one skilled in the art.

The therapeutic compositions are administered to a subject diagnosed with cancer or is suspected of having a cancer. Subjects may be human or non-human animals.

The compositions are used to treat cancer. The terms “treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

Representative forms of cancer include carcinomas, leukemia's, lymphomas, myelomas and sarcomas. Further examples include, but are not limited to cancer of the bile duct cancer, brain (e.g., glioblastoma), breast, cervix, colorectal, CNS (e.g., acoustic neuroma, astrocytoma, craniopharyogioma, ependymoma, glioblastoma, hemangioblastoma, medulloblastoma, menangioma, neuroblastoma, oligodendroglioma, pinealoma and retinoblastoma), endometrial lining, hematopoietic cells (e.g., leukemia's and lymphomas), kidney, larynx, lung, liver, oral cavity, ovaries, pancreas, prostate, skin (e.g., melanoma and squamous cell carcinoma) and thyroid. Cancers can comprise solid tumors (e.g., sarcomas such as fibrosarcoma, myxosarcoma, liposarcoma, chondrosarcoma and osteogenic sarcoma), be diffuse (e.g., leukemia's), or some combination of these (e.g., a metastatic cancer having both solid tumors and disseminated or diffuse cancer cells). Cancers can also be resistant to conventional treatment (e.g. conventional chemotherapy and/or radiation therapy).

Benign tumors and other conditions of unwanted cell proliferation may also be treated.

The oHSV as described herein may be given by a route that is e.g., oral, topical, parenteral, systemic, intravenous, intramuscular, intraocular, intrathecal, intratumor, subcutaneous, or transdermal. Within certain embodiments the oncolytic virus may be delivered by a cannula, by a catheter, or by direct injection. The site of administration may be intra-tumor or at a site distant from the tumor. The route of administration will often depend on the type of cancer being targeted.

The optimal or appropriate dosage regimen of the oncolytic virus is readily determinable within the skill of the art, by the attending physician based on patient data, patient observations, and various clinical factors, including for example a subject's size, body surface area, age, gender, and the particular oncolytic virus being administered, the time and route of administration, the type of cancer being treated, the general health of the patient, and other drug therapies to which the patient is being subjected. According to certain embodiments, treatment of a subject using the oncolytic virus described herein may be combined with additional types of therapy, such as chemotherapy using, e.g., a chemotherapeutic agent such as etoposide, ifosfamide, adriamycin, vincristin, doxicyclin, and others.

oHSV may be formulated as medicaments and pharmaceutical compositions for clinical use and may be combined with a pharmaceutically acceptable carrier, diluent, excipient or adjuvant. The formulation will depend, at least in part, on the route of administration. Suitable formulations may comprise the virus and inhibitor in a sterile medium. The formulations can be fluid, gel, paste or solid forms. Formulations may be provided to a subject or medical professional

A therapeutically effective amount is preferably administered. This is an amount that is sufficient to show benefit to the subject. The actual amount administered and time-course of administration will depend at least in part on the nature of the cancer, the condition of the subject, site of delivery, and other factors.

Within yet other embodiments of the invention the oncolytic virus can be administered intratumorally, or, after surgical resection of a tumor.

The following examples are offered by way of illustration, and not by way of limitation.

### EXAMPLES

### Example 1

### Enhancement of Viral Production

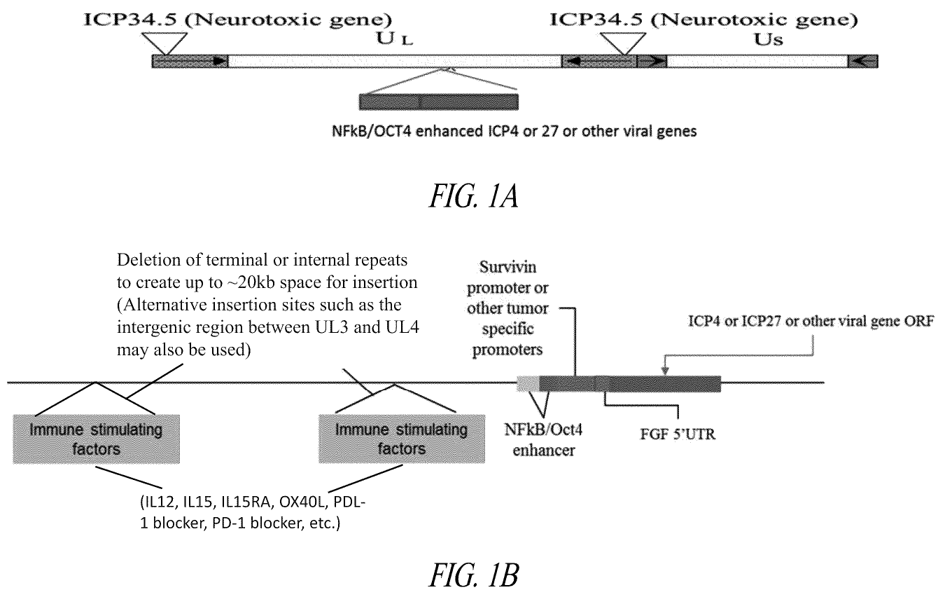

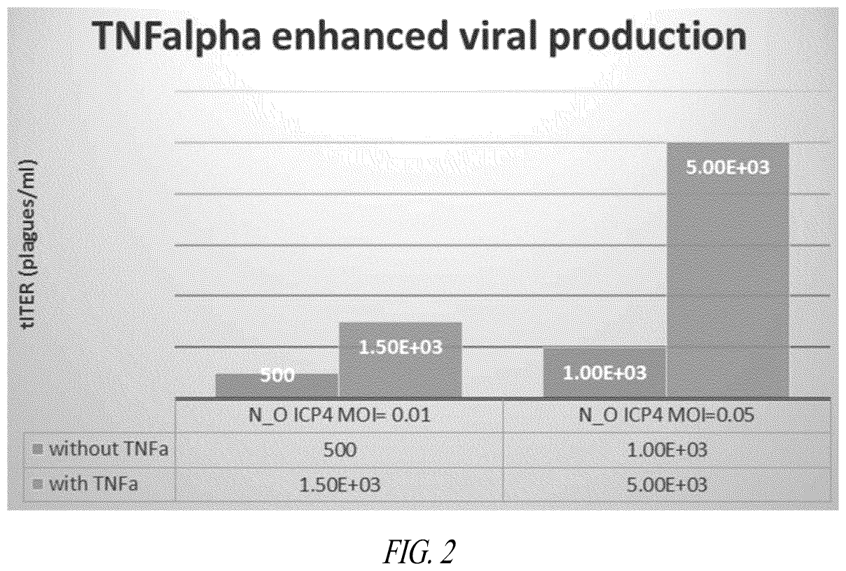

In this example, the production of oHSV-1 shown in FIG. 1A was measured in the presence of an activator of NF-κB. The virus has a NF-κB response element in the regulatory region of ICP4. Viral production was significantly enhanced in the presence of TNFalpha that activates NF-κB (FIG. 2).

### Example 2

### Exemplary Constructs

In this example, various constructs and their sequences are presented.

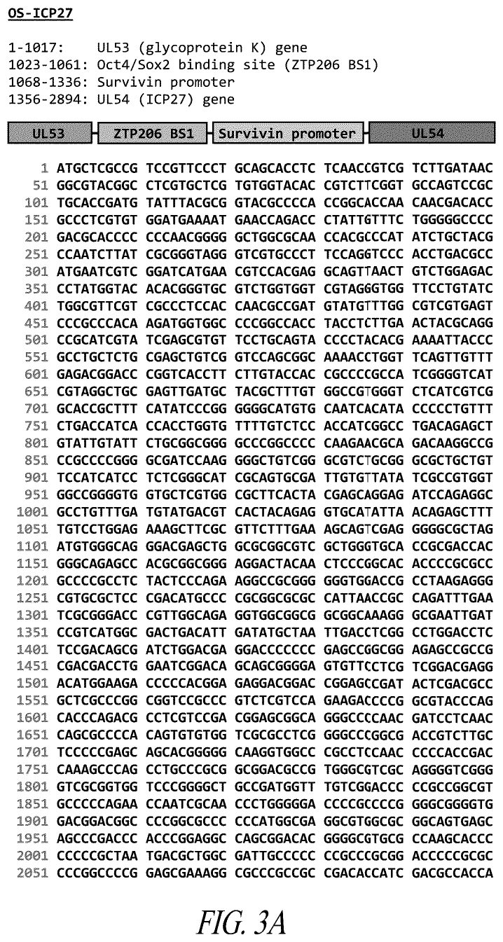

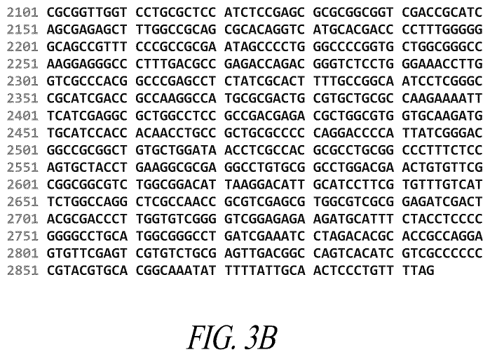

OS-ICP27 comprises ZTP206 BSI (Oct4/Sox2 binding site) and a survivin promoter which replace the native intergenic region between UL53 (glycoprotein K) and UL54 (ICP27) (FIG. 3).

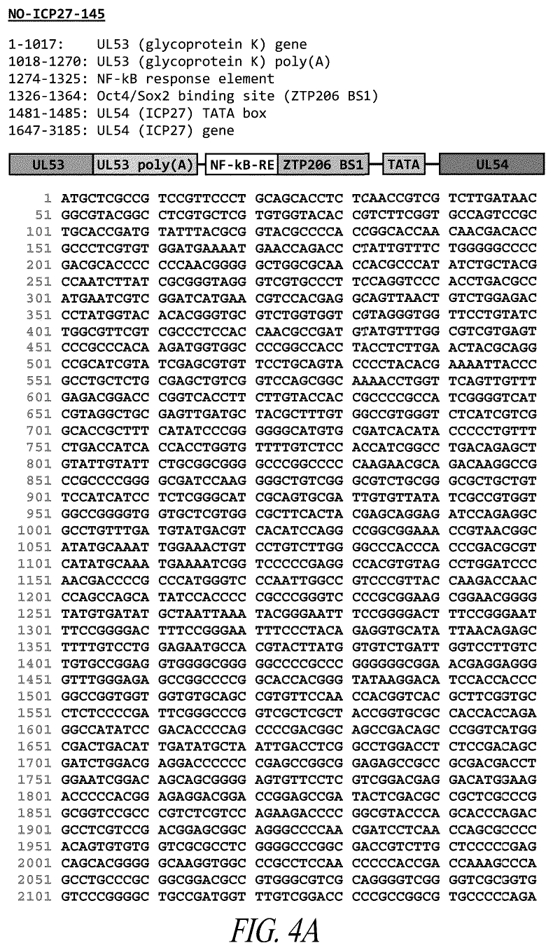

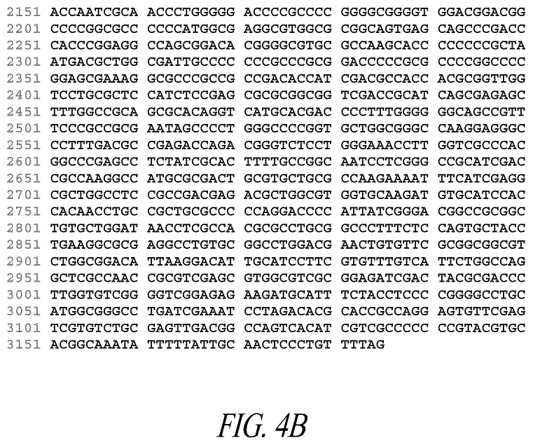

NO-ICP27-145 comprises an NF-kB response element and Oct4/Sox2 binding site (ZTP206 BSI) inserted within the intergenic region between UL53 (glycoprotein K) and UL54 (ICP27) at a location downstream of the UL53 (glycoprotein K) poly(A) and upstream of the UL54 (ICP27) TATA box. (FIG. 4).

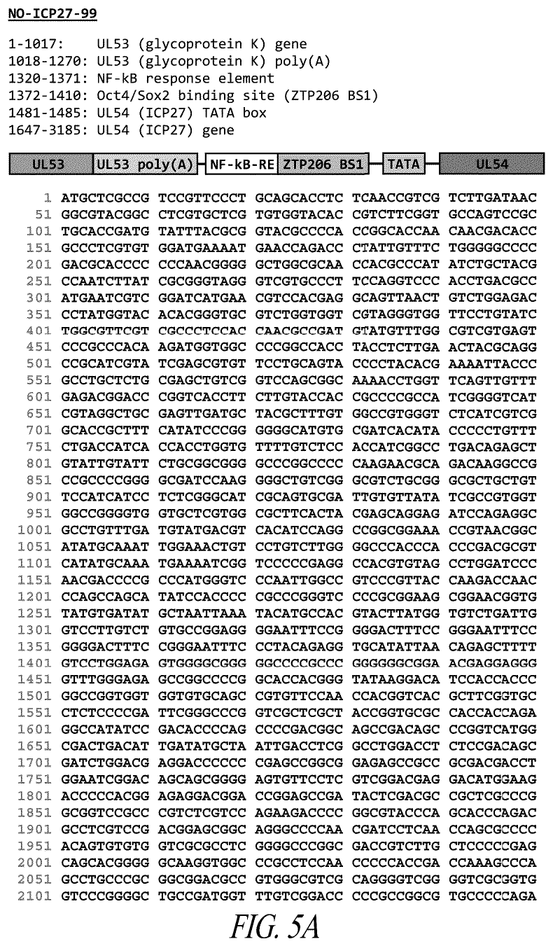

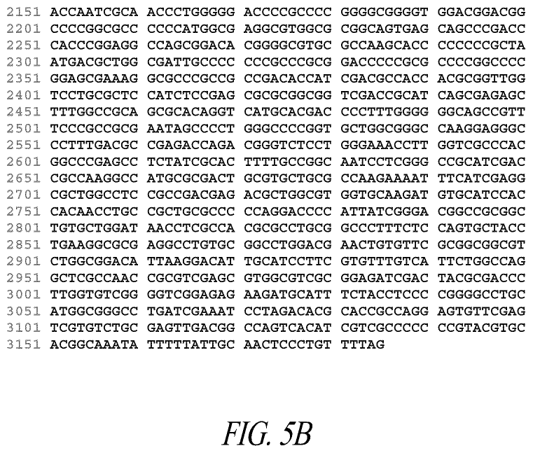

NO-ICP27-99 comprises an NF-kB response element and Oct4/Sox2 binding site (ZTP206 BSI) inserted within the intergenic region between UL53 (glycoprotein K) and UL54 (ICP27) at a location downstream of the UL53 (glycoprotein K) poly(A) and upstream of the UL54 (ICP27) TATA box. (FIG. 5).

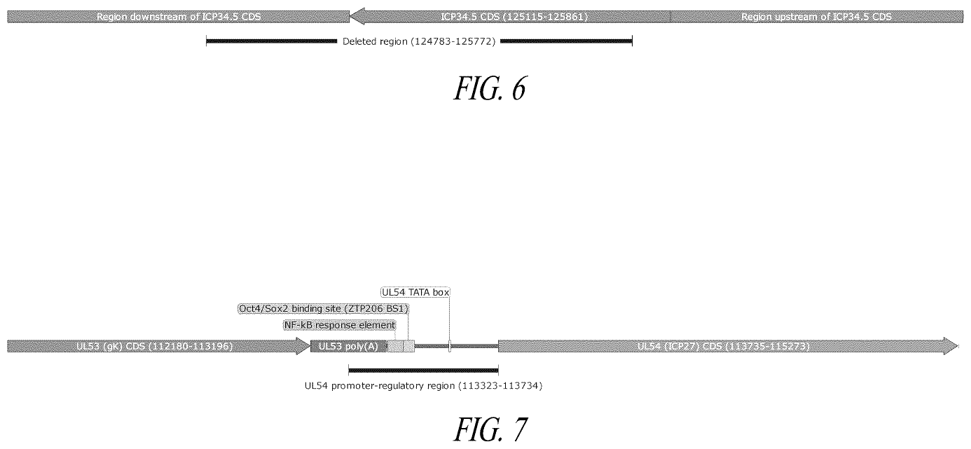

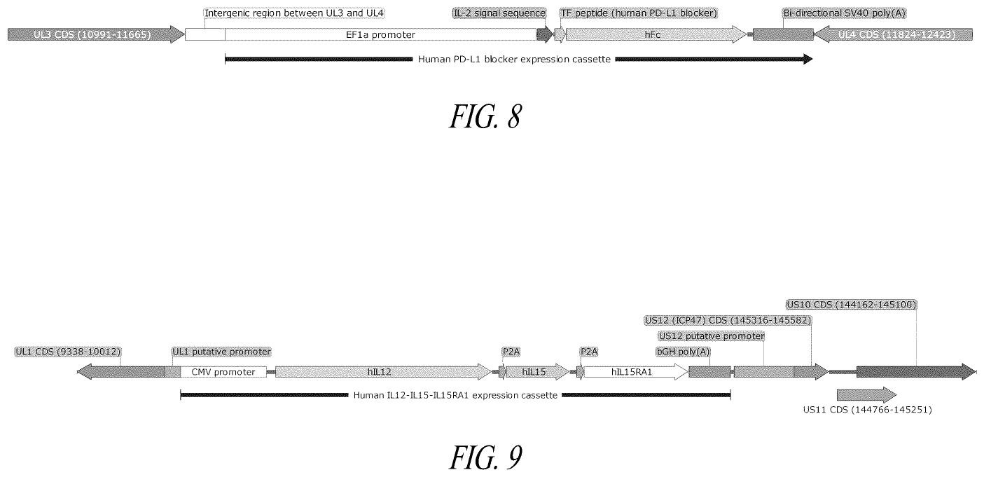

hVG161 comprises a modified ICP34.5 region (FIG. 6; SEQ ID NO. 6), a modified UL54 promoter-regulatory region (FIG. 7; SEQ ID NO. 7), an insertion of a PD-L1 blocker within the intergenic region between UL3 and UL4 (FIG. 8; SEQ ID No. 8), and a modified terminal repeat (TR) region carrying an expression cassette encoding IL-12, IL-15, and IL-15 receptor alpha subunit (FIG. 9; SEQ ID NO. 9). These four viruses also have a modified and partially deleted ICP 34.5 region.

mVG161 is a functionally identical mouse version of hVG161 except that that mVG161 carries a mouse version of IL-12 and a mouse PD-L1 blocker in the same location on the viral genome where hVG161 carries a human IL-12 and a human PD-L1 blocker.

### Example 3

### Abbreviations Used in Subsequent Examples

TF-Fc: PD-L1 blocking peptide (TF) fused to Fc and used for construction of VG161.

IL-TF-Fc: plasmid carrying IL-12, IL-15, and PD-L1 blocker.

HSV-345: ICP34.5-deleted virus.

OS-ICP27 2-11: ICP34.5-deleted virus with Oct4/Sox2 binding site and surviving promoter (OS) inserted within the promoter-regulatory region of ICP27 (OS-1CP27) that was not used for construction of VG161.

OS-ICP27 5-7: ICP34.5-deleted virus with OS-ICP27 mutation that was not used for construction of VG161.

NO-ICP27 1-4-4 (also known as NO-ICP27-145): ICP34.5-deleted virus with NF-kB response element and Oct4/Sox2 binding site (NO) inserted within the promoter-regulatory region of ICP27 (NO-ICP27) at a location 145 bp upstream of the transcription start site of ICP27 and that was used for construction of VG161.

NO-ICP27 5-2-2 (also known as NO-ICP27-99): ICP34.5-deleted virus with NF-kB response element and Oct4/Sox2 binding site (NO) inserted within the promoter-regulatory region of ICP27 (NO-ICP27) at a location 99 bp upstream of the transcription start site of ICP27 and that was not used for construction of VG161.

VG001 (also known as VG160): backbone virus that was used for construction of VG161 (NO-ICP27 1-4-4 mutant carrying an exogenous promoter and poly(A) flanking an empty MCS within deleted terminal repeat region of the viral genome that is subsequently used for insertion of the IL-12/IL-15 expression cassette).

VG001-15h (also known as VG161-15h): VG001 carrying human IL-15.

VG001-1215h (also known as VG161-1215h): VG001 carrying human IL-12 and human IL-15.

VG001-PLBh (also known as VG161-PLBh): VG001 carrying human PD-L1 blocker inserted within intergenic region between UL3 and UL4.

8-8-15RA1-PDL1b: VG001 carrying human IL-15 and human PD-L1 blocker.

VG161-1215PLBm (also known as mVG161): VG001 carrying mouse IL-12, human IL-15, and mouse PD-L1 blocker.

VG161-1215PLBh (also known as hVG161 or VG161): VG001 carrying human IL-12, human IL-15, and human PD-L1 blocker.

### Example 4

### Results of Exemplary Constructs Comprising Mutations in the Icp27 Promoter/Regulatory Region

In this example, viruses with mutations in the ICP27 promoter-regulatory region were used to infect cells.

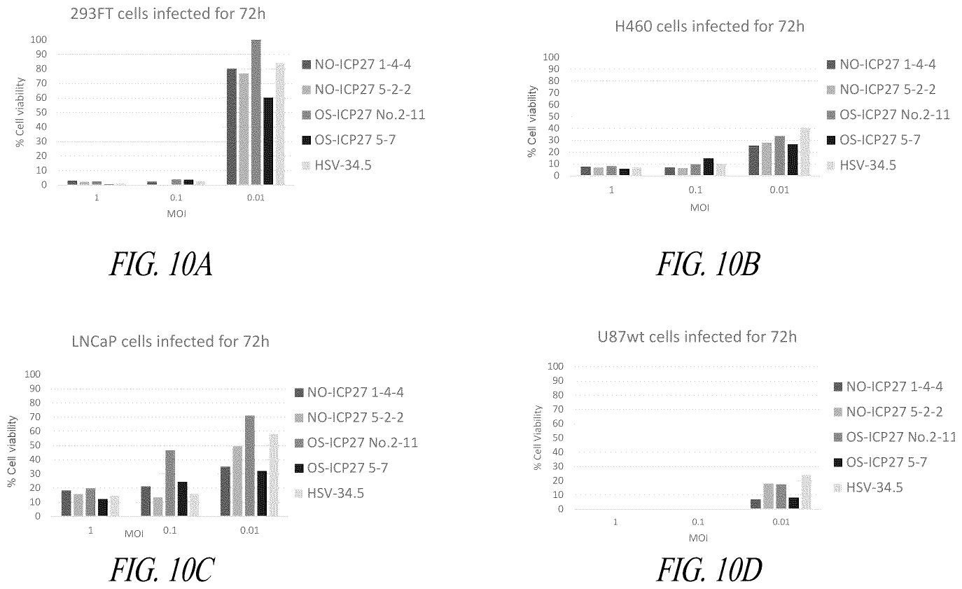

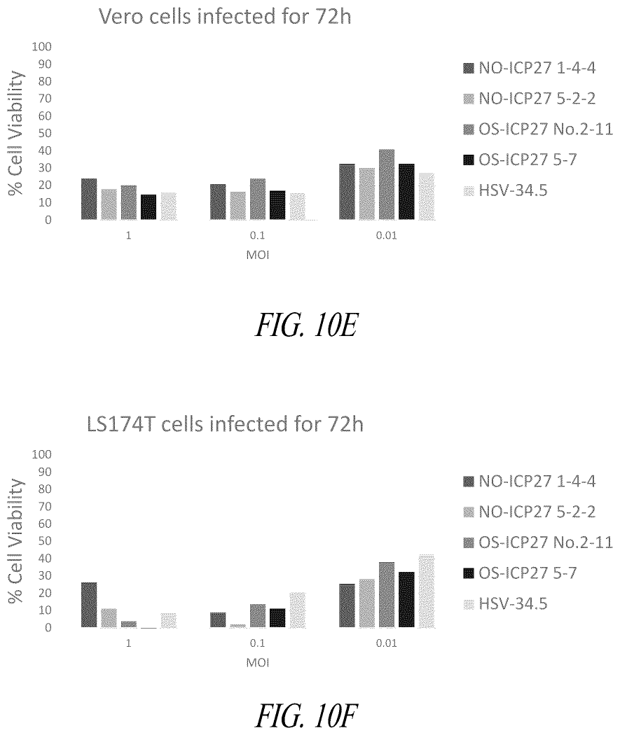

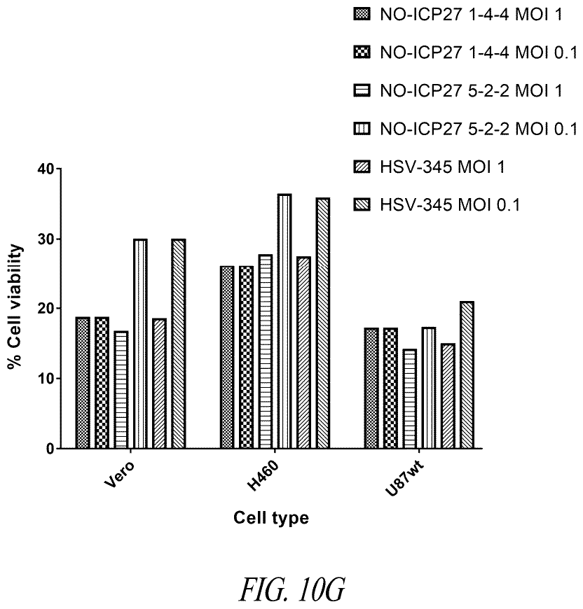

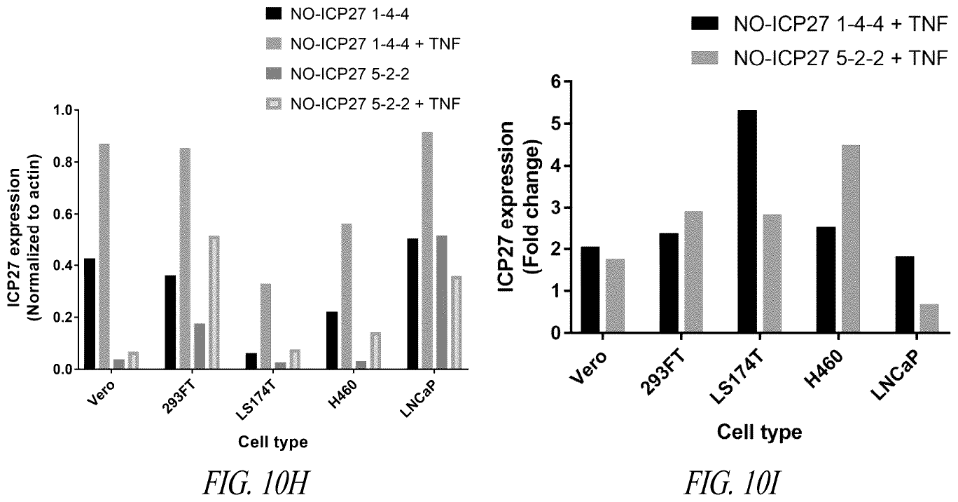

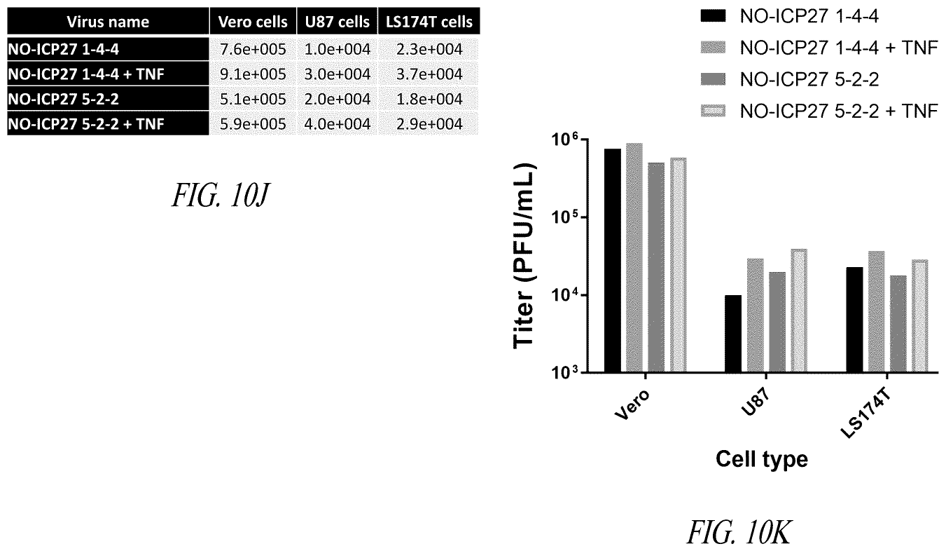

In FIG. 10A-G, Vero, LS174T, 293FT, H460, U87 wt, and LNCaP cells were infected with the indicated HSV-1 mutants at MOI 0.01, 0.1, and 1. Cell viability was quantified using MTT assay at 72 hours post infection.

In FIG. 10H-I, Vero, 293FT, LS174T, H460, and LNCaP cells were infected with HSV-1 mutants NO-ICP27 1-4-4 and NO-ICP27 5-2-2 at MOI 1 both with and without induction using 20 ng/mL of TNFα. Infected cells were harvested 5 hours post-infection and the cell lysates were probed with anti-ICP27 and anti-actin antibodies to detect expression of ICP27 and actin, respectively. Actin was used as a loading control for the western blot. Band intensity was quantified and used to estimate expression of ICP27 normalized to actin (FIG. 10H). TNFα-mediated induction of ICP27 expression was measured (FIG. 10I) as fold change in normalized ICP27 expression following treatment with TNFα. A robust response to TNFα was evident in both versions of NO-ICP27 but the NO-ICP27 1-4-4 variant was ultimately selected for inclusion in the VG161 mutant virus due to stronger upregulation of ICP27 expression.

In FIG. 10J-K, Vero, U87, and LS174T cells were infected with HSV-1 mutants NO-ICP27 1-4-4 and NO-ICP27 5-2-2 at MOI 0.1 both with and without induction using 20 ng/mL of TNFα. Cells were harvested 24 hours post-infection and subjected to one freeze/thaw cycle to completely lyse the infected cells prior to titration on Vero cells. FIG. 10J shows the raw titer data while FIG. 10K is a graphical representation of the data from FIG. 10J. Both NO-ICP27 mutants exhibit enhanced replication on Vero, U87, and LS174T cells after TNFα induction. The NO-ICP27 1-4-4 mutant was ultimately selected as a backbone for insertion of the PD-L1 blocker and the IL-12/IL-15 expression cassette to create the final VG161 mutant virus.

### Example 5

### Expression of IL-12 Following Infection of Cells by hVG161

In this Example, Western blot and ELISA data of 11-12 expression is shown.

FIG. 11A shows Western Blot results after VG161-1215PLBh virus infection. H460 tumour cells were infected with VG161-1215PLB or VG001 virus (MOI=1) for 24 hours. Cell lysates were prepared, ran on 12% SDS-PAGE gel, and transferred to PVDF membrane. The membrane was blotted with anti-human IL-12 antibody followed by HRP-conjugated anti-mouse IgG secondary antibody and the mage was detected and analyzed using Bio-Rad ImageLab system.

FIG. 11B-C shows that production of human IL-12 is upregulated after VG161-1215PLBh virus infection. LS174T or H460 tumour cells were infected with VG161-1215PLB or VG001 virus (MOI=1) for 48 hours. Infected cell supernatants were harvested and bound to anti-human IL-12 capture antibody coated 96-well Immuno Maxisorp flat bottom plate. Binding was detected via a biotinylated anti-human IL-12 antibody, avidin-horseradish peroxidase (HRP), and 3,3′,5,5′-Tetramethylbenzidine (TMB) substrate. Absorbance measurements were collected at 450 nm via a plate reader. The concentration of human IL-12 in cultured supernatants was calculated based on human IL-12 standard curve.

### Example 6

### Expression of IL-15 Following Infection of Cells by hVG161

In this Example, Western blot and ELISA data of IL-15 expression is shown.

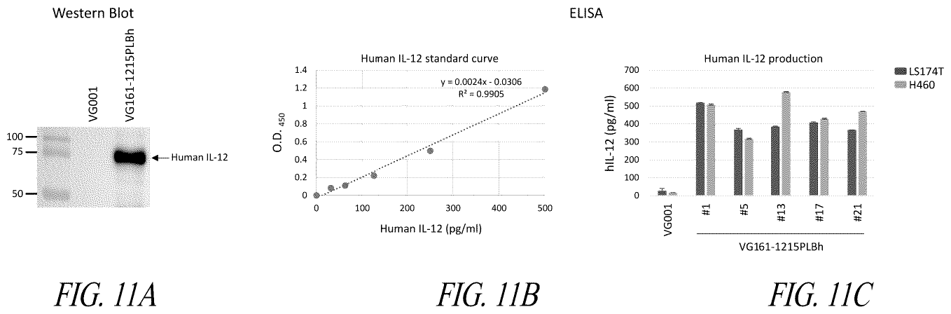

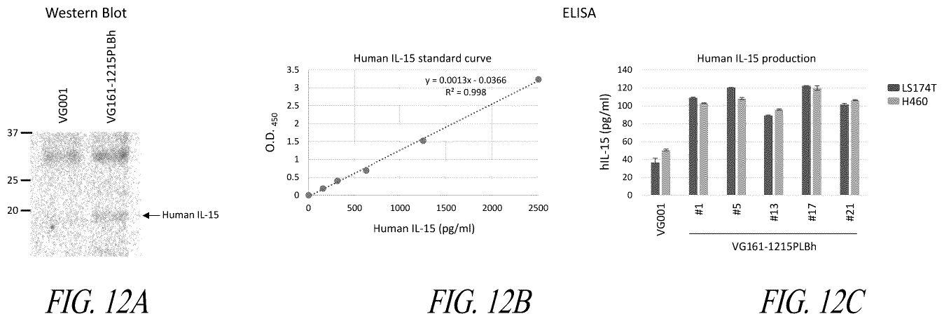

FIG. 12A shows Western Blot results after VG161-1215PLBh virus infection. H460 tumour cells were infected with VG161-1215PLB or VG001 virus (MOI=1) for 24 hours. Cell lysates were prepared, ran on 12% SDS-PAGE gel, and transferred to PVDF membrane. The membrane was blotted with anti-human IL-15 antibody followed by HRP-conjugated anti-mouse IgG secondary antibody and the image was detected and analyzed using Bio-Rad ImageLab system.

FIG. 12B-C shows that production of human IL-15 is upregulated after VG161-1215PLBh virus infection. LS174T or H460 tumour cells were infected with VG161-1215PLB or VG001 virus (MOI=1) for 48 hours. Infected cell supernatants were harvested and bound to anti-human IL-15 capture antibody coated 96-well Immuno Maxisorp flat bottom plate. Binding was detected via a biotinylated anti-human IL-15 antibody, avidin-horseradish peroxidase (HRP), and 3,3′,5,5′-Tetramethylbenzidine (TMB) substrate. Absorbance measurements were collected at 450 nm via a plate reader. The concentration of human IL-15 in cultured supernatants was calculated based on human IL-15 standard curve.

### Example 7

### Expression of IgG4 Following Infection of Cells by hVG161

In this Example, Western blot and ELISA data of IgG4 expression is shown.

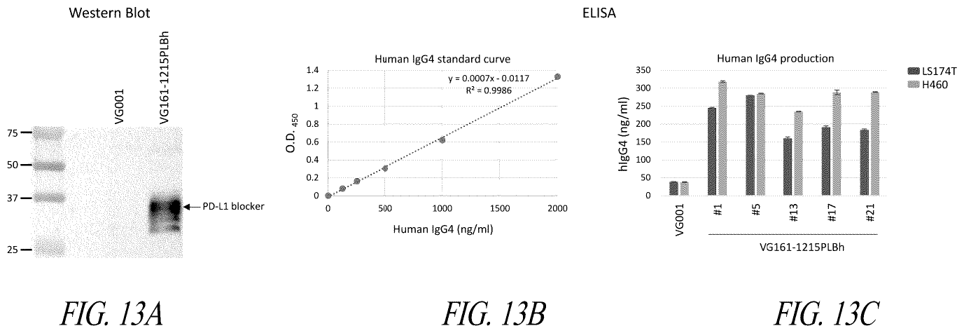

FIG. 13A shows Western Blot results after VG161-1215PLBh virus infection. H460 tumour cells were infected with VG161-1215PLB or VG001 virus (MOI=1) for 24 hours. Cell lysates were prepared, ran on 12% SDS-PAGE gel, and transferred to PVDF membrane. The membrane was blotted with HRP-conjugated anti-human IgG antibody and the image was detected and analyzed using Bio-Rad ImageLab system.

FIG. 13B-C shows that production of human PD-L1 blocker (fused to human Fc domain) is upregulated after VG161-1215PLBh virus infection. LS174T or H460 tumour cells were infected with VG161-1215PLB or VG001 virus (MOI=1) for 48 hours. Infected cell supernatants were harvested and bound to anti-human IgG4 capture antibody coated 96-well Immuno Maxisorp flat bottom plate. Binding was detected via a biotinylated anti-human IgG4 antibody, avidin-horseradish peroxidase (HRP), and 3,3′,5,5′-Tetramethylbenzidine (TMB) substrate. Absorbance measurements were collected at 450 nm via a plate reader. The concentration of human IgG4 in cultured supernatants was calculated based on human IgG4 standard curve.

### Example 8

### In Vitro Efficacy of Viruses VG161-pLBH and VG161-15h

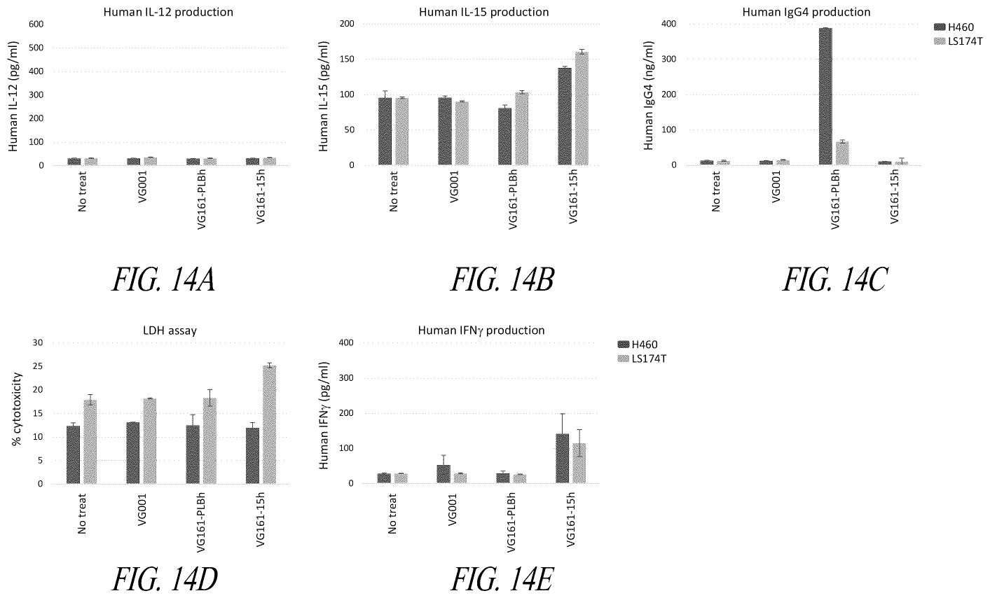

In this example, 3×10⁴ H460 or LS174T tumour cells were seeded into each well of 96-well plate and cultured at 37° C. for overnight. Next day, seeded cells were infected with VG001 backbone, VG161-PLBh, or VG161-15h virus (MOI=1) for 24 hours and the productions of human IL-12, human IL-15, and human IgG4 were assessed (FIG. 14A-C). 3×10⁵ human PBMCs were subsequently added into the culture and co-incubated for 24 hour to assess cytotoxicity by LDH assay (FIG. 14D) or 48 hours for human IFNg production by ELISA (FIG. 14E). For the cytotoxicity assay, percentage of cytotoxicity was calculated based on the following formula: [(actual reading−minimum release)/(Maximum release−minimum release)]×100%. Supernatant harvested from tumour cells incubated with medium only was used as minimum release, and supernatant harvested from tumour cells incubated with lysis buffer was used as maximum release.

### Example 9

### In Vitro Efficacy of Various Constructs

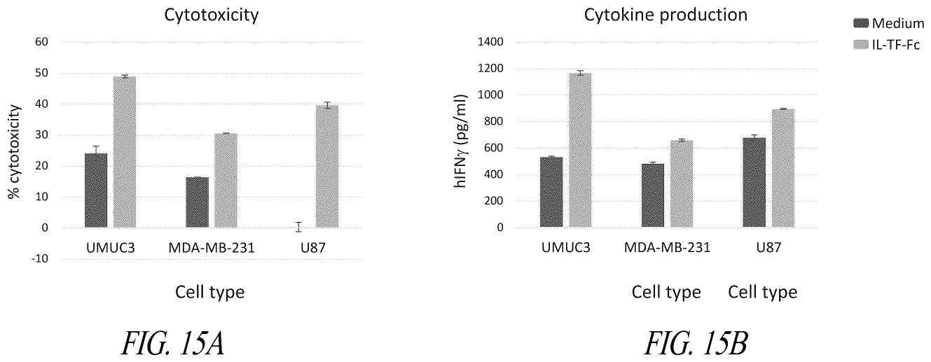

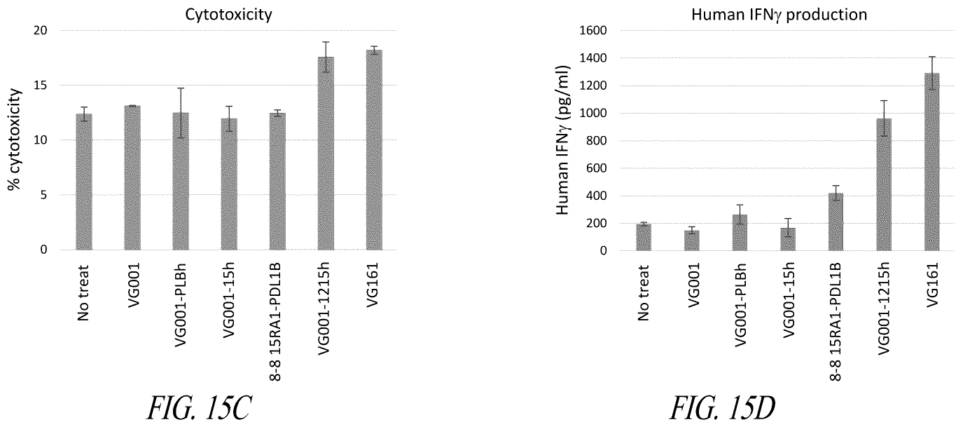

FIGS. 15A-15D show results of in vitro assays for various constructs.

FIG. 15A-15B show results of cell transfection with IL-TF-Fc plasmid carrying IL-12, IL-15, and PD-L1 blocker. In FIGS. 15A-15B, different tumour cell lines were transfected with IL-TF-Fc plasmid DNA for 24 hours, and human PBMCs were subsequently added into the culture. Cell supernatants were harvested after 24 hours for quantification of cytotoxicity by LDH assay (FIG. 15A), and after 48 hours for detection of human IFNg production by ELISA assay (FIG. 15B).

FIGS. 15C-15D show results of cell infection with a variety of mutant viruses including hVG161. Virally encoded IL12, IL15, and PD-L1 blocker synergistically enhance IFNg production and cytotoxicity. H460 tumour cells were seeded into each well of a 96-well plate and cultured at 37° C. for overnight. Next day, seeded cells were infected with the indicated viruses at MOI=1 for 24 hours. Human PBMCs were subsequently added into the culture and co-incubated for 24 hour to assess cytotoxicity by LDH assay (FIG. 15C) or 48 hours for human IFNg production by ELISA (FIG. 15D). For cytotoxicity assay, the percentage of cytotoxicity was calculated based on the following formula: [(actual reading−minimum release)/(Maximum release−minimum release)]×100%. Supernatant harvested from tumour cells incubated with medium only was used as minimum release, and supernatant harvested from tumour cells incubated with lysis buffer was used as Maximum release.

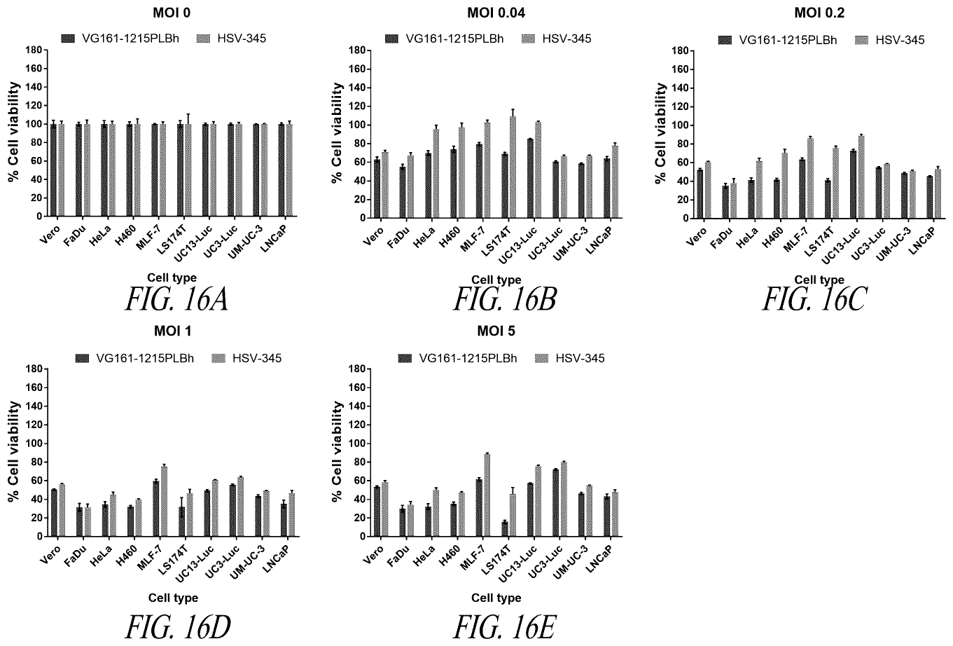

In FIGS. 16A-16E, a panel of 9 different human tumor cell lines (plus Vero cells) was infected with VG161-1212PLBh (VG161h) and HSV-345 viruses at MOI 0, 0.04, 0.2, 1, and 5. Cell viability was quantified using MTT assay at 48 hours post infection.

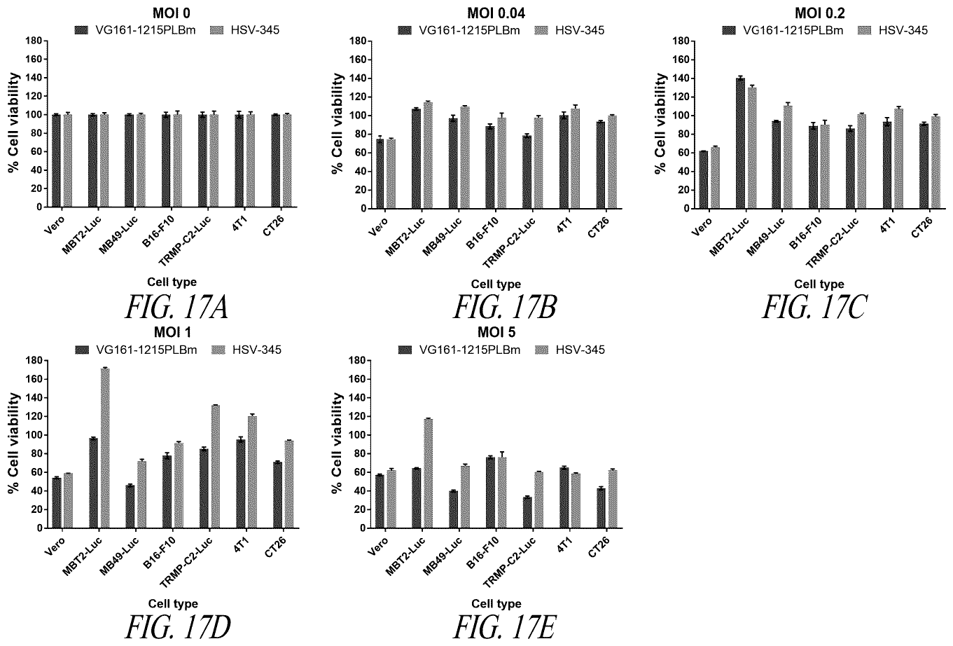

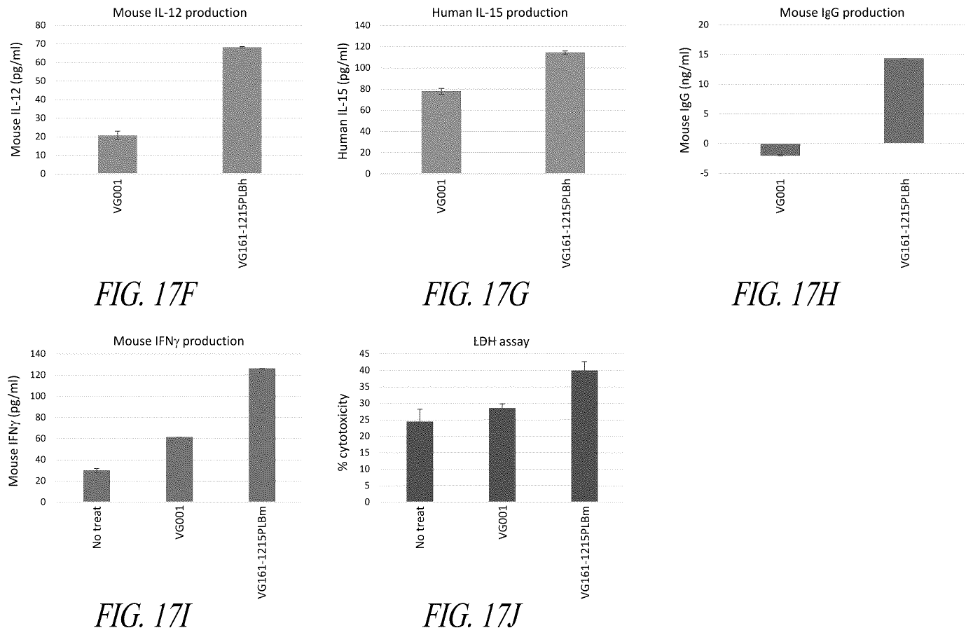

FIGS. 17A-17J show results of in vitro assays for various constructs. FIGS. 17A-17E show results of cell viability assays for mVG161 and HSV-345 on mouse tumor cell lines and Vero cell line; FIGS. 17F-17J show the characterization of transgene expression following mVG161 or VG001 infection of CT26 mouse tumor cells.

In FIGS. 17A-17E, a panel of 6 different mouse tumor cell lines (plus Vero cells) was infected with VG161m and HSV-345 viruses at MOI 0, 0.04, 0.2, 1, and 5. Cell viability was quantified using MTT assay at 48 hours post infection.

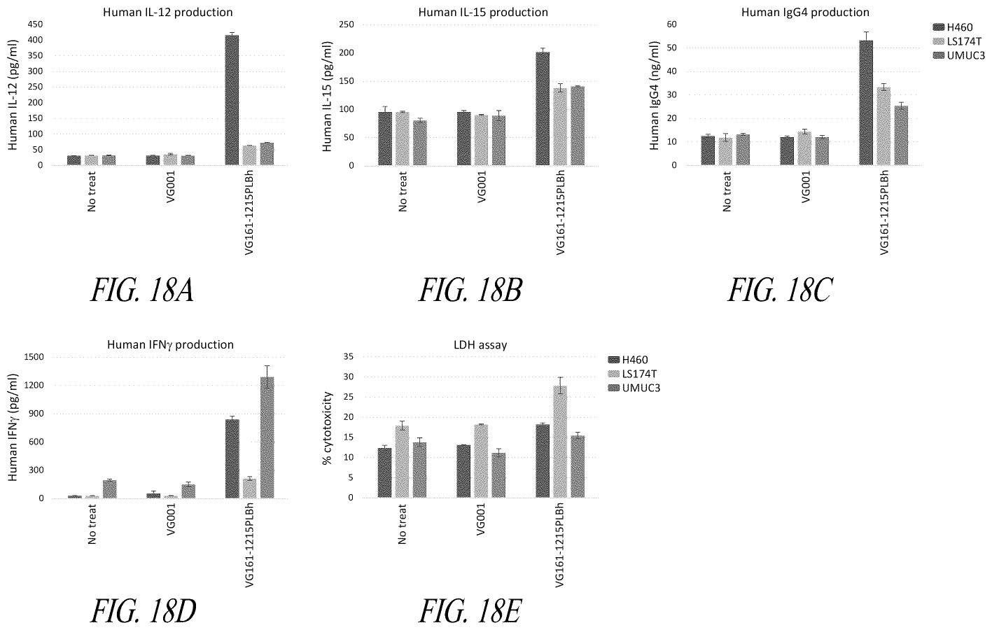

In FIGS. 17F-17J, 3×10⁴ CT26 tumour cells were seeded into each well of 96-well plate and cultured at 37° C. for overnight. Next day, seeded cells were infected with VG001 backbone or VG161-1215PLBm virus (MOI=1) for 24 hours and the production of mouse IL-12, human IL-15, and mouse IgG was assessed (FIG. 18O). 3×10⁵ splenocytes from Balb/c mouse were subsequently added into the culture and co-incubated for 24 hour to assess cytotoxicity by LDH assay (FIG. 18P) or 48 hours for mouse IFNg production by ELISA (FIG. 18O). For the cytotoxicity assay, percentage of cytotoxicity was calculated based on the following formula: [(actual reading−minimum release)/(Maximum release−minimum release)]×100%. Supernatant harvested from tumour cells incubated with medium only was used as minimum release, and supernatant harvested from tumour cells incubated with lysis buffer was used as maximum release.

In FIGS. 18A-18E, 3×10⁴ H460, LS174T, or UMUC3 tumor cells were seeded into each well of 96-well plate and cultured at 37° C. for overnight. Next day, seeded cells were infected with VG001 backbone and VG161-1215h virus (MOI=1) for 24 hours and the productions of human IL-12, human IL-15, and human IgG4 were assessed (18R). 3×10⁵ human PBMCs were subsequently added into the culture and co-incubated for 24 hour to assess cytotoxicity by LDH assay (18S) or 48 hours for human IFNγ production by ELISA (18T). For the cytotoxicity assay, percentage of cytotoxicity was calculated based on the following formula: [(actual reading−minimum release)/(Maximum release−minimum release)]×100%. Supernatant harvested from tumor cells incubated with medium only was used as minimum release, and supernatant harvested from tumor cells incubated with lysis buffer was used as maximum release.

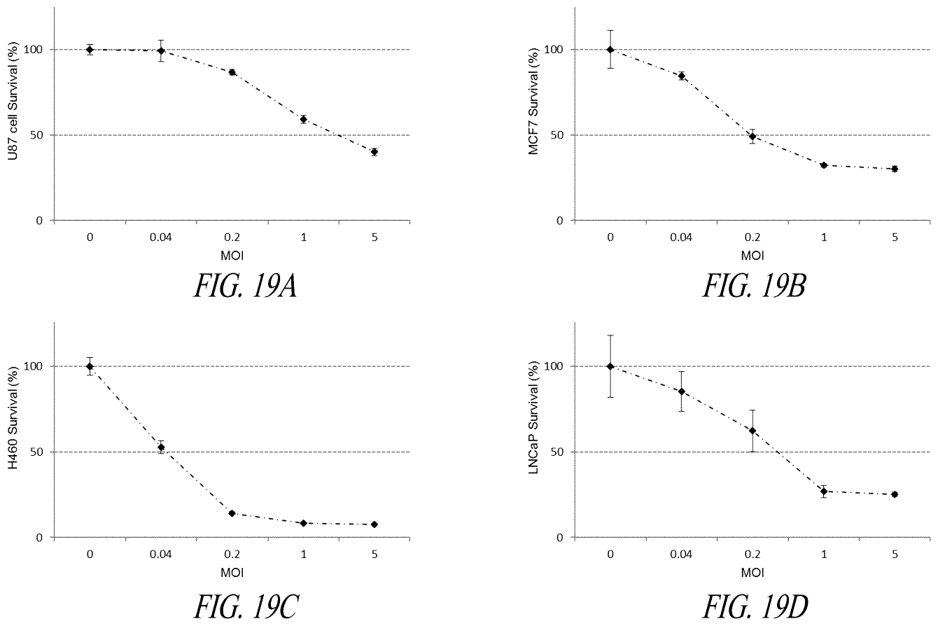

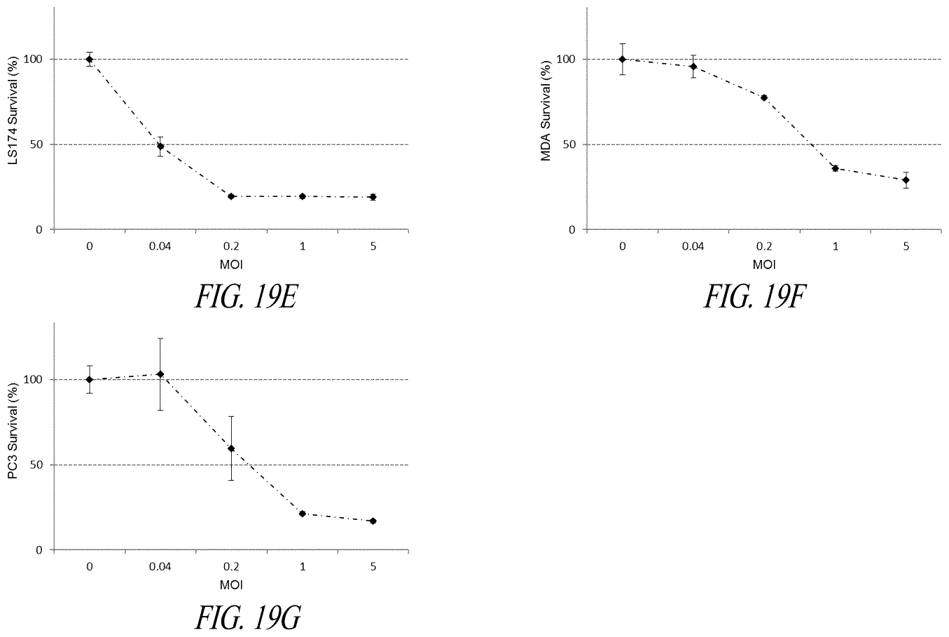

In FIG. 19A-19G, the antitumor effect of VG161-1215PLBh (hVG161) virus was evaluated in a variety human cancer cells including U87, MCF7, H460, LNCaP, LS174T, MDA, and PC3 at 72h post infection and MOIs ranging from 0 to 5. Cell survival percentage was quantified by MTT assay. The VG161-1215PLBh virus exhibits robust cell killing ability in all of the tested human tumor cell lines.

### Example 11

### In Vivo Efficacy of VG161 Viral Constructs

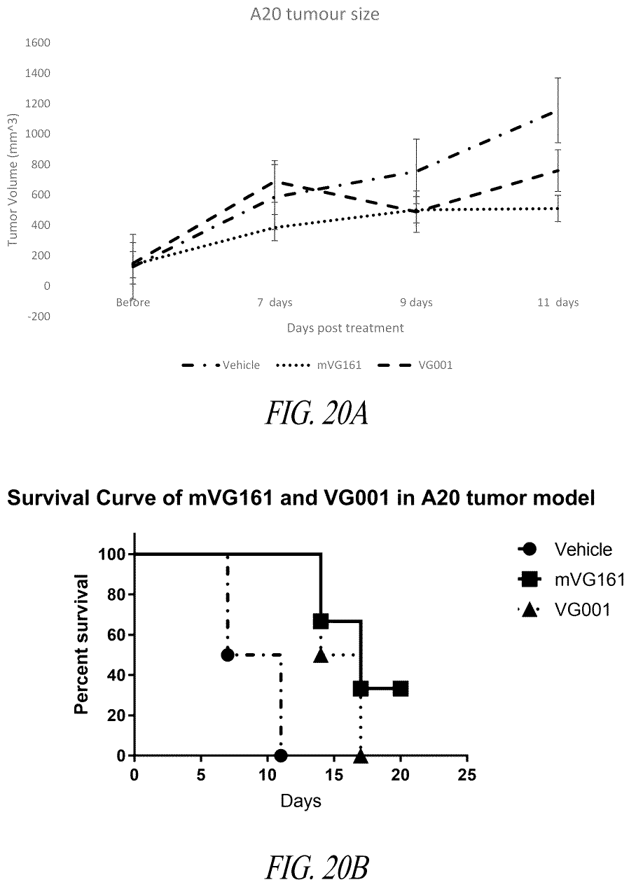

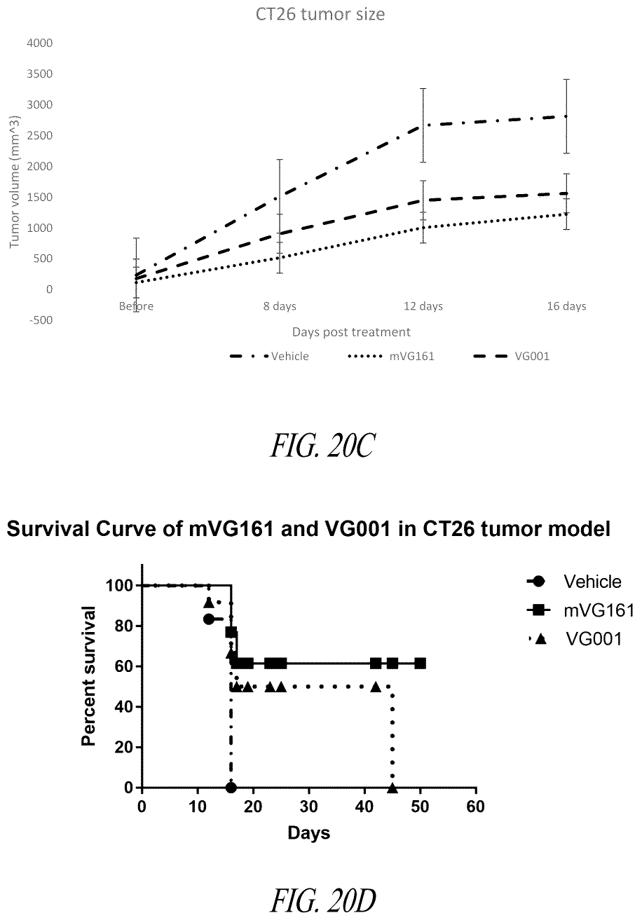

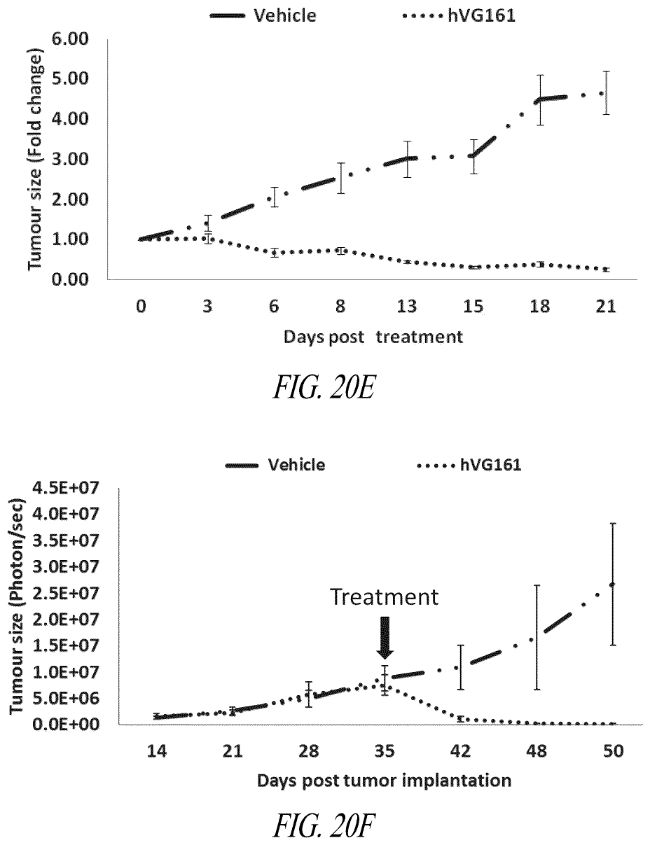

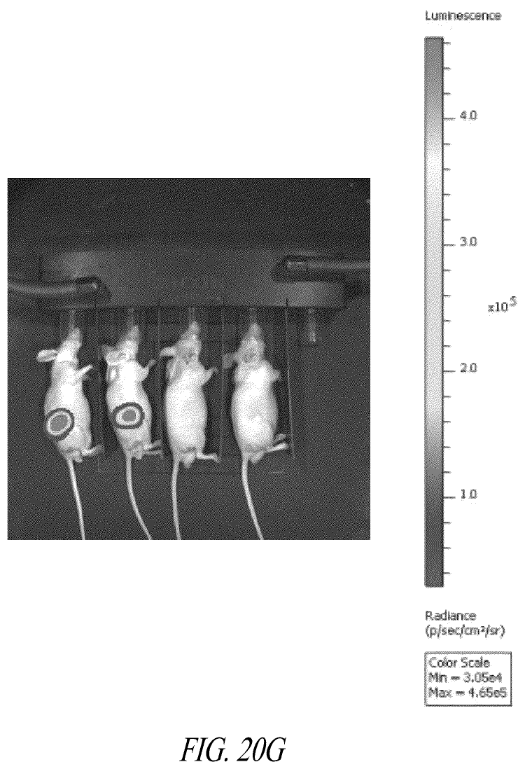

In FIG. 20A-B, BALB/c mice bearing A20 murine B-cell lymphoma tumors were injected 5 times intratumorally with a total of 1×10\hat{ }7 PFU/mouse of either VG161-1215PLBm (mVG161) virus or VG001 backbone virus or with PBS (vehicle control). Tumor size measurements were performed at the indicated times post injection. Mice treated with VG161-1215PLBm exhibited a significant (P<0.05) reduction in tumor volume compared to mice treated with PBS.

In FIGS. 20C-D, BALB/c mice bearing CT26 murine colon carcinoma tumors were injected 5 times intratumorally with a total of 5×10\hat{ }6 PFU/mouse of either VG161-1215PLBm (mVG161) virus or VG001 backbone virus or with PBS (vehicle control). Tumor size measurements were performed at the indicated times post injection. Mice treated with VG161-1215PLBm exhibited a significant (P<0.05) reduction in tumor volume compared to mice treated with PBS.

In FIGS. 20E-G, oHSV treatment of xenograft human prostate tumors in mice was assessed. Twelve mice were implanted with LNCaP human prostate tumor cells in the right lower flank. At 35 days post implantation, a randomly selected group of 6 animals was injected twice intratumorally with a total of 5×10\hat{ }7 PFU/mouse of VG161-1215PLBh (hVG161) virus, while the remaining 6 animals served as a vehicle control and were injected twice with an equivalent volume of PBS. Tumor size measurements were performed using two different methods. Caliper measurements are expressed as fold change in tumor volume at a given time point compared to the tumor volume at the time of virus or PBS injection (FIG. 20E). Tumor-bearing mice treated with VG161-1215PLBh virus exhibited robust tumor shrinkage during the course of the study with over 50% reduction in tumor size at the end of 15 days, while vehicle-treated mice showed approximately 3-fold increases in tumor volume during the same time span. Tumor growth was also monitored using a whole animal bioluminescent imaging system (IVIS Imaging System; Xenogen, Mountain View, CA). Signal intensities were quantified as the sum of all detected photons per second (FIG. 20F). Quantitative imaging of tumor growth using the IVIS system shows an even more dramatic reduction in tumor size in oHSV-treated animals compared to PBS-treated controls, with fluorescence dropping to undetectable levels by 50 days post tumor implantation (FIG. 20G; two vehicle controls on left and two oHSV-treated mice on right).

### Example 12

### Replication of hVG161 in Cell Lines

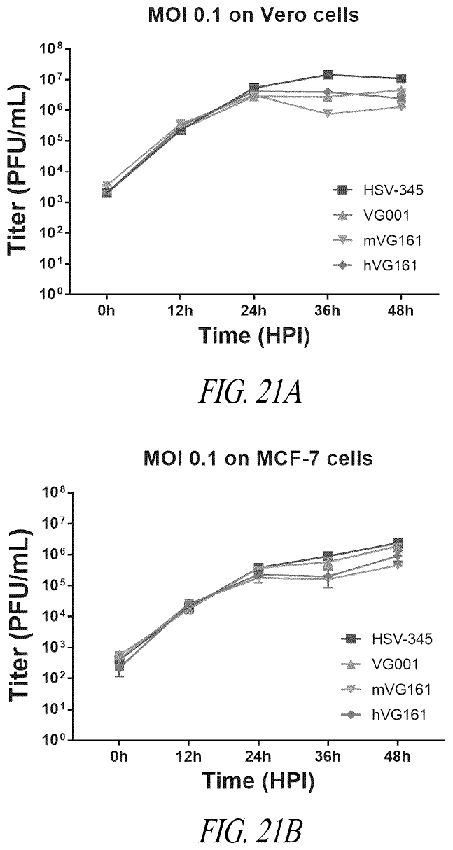

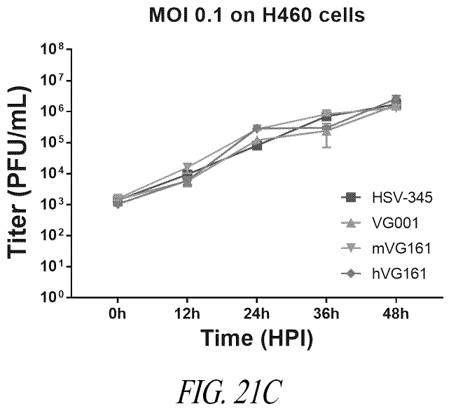

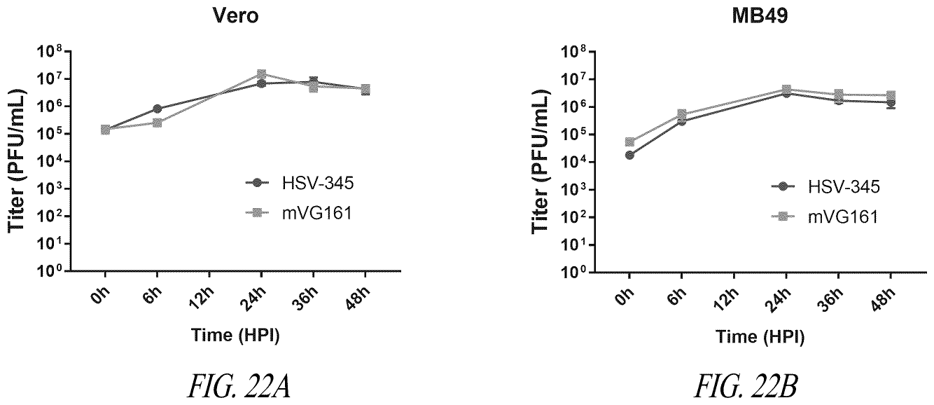

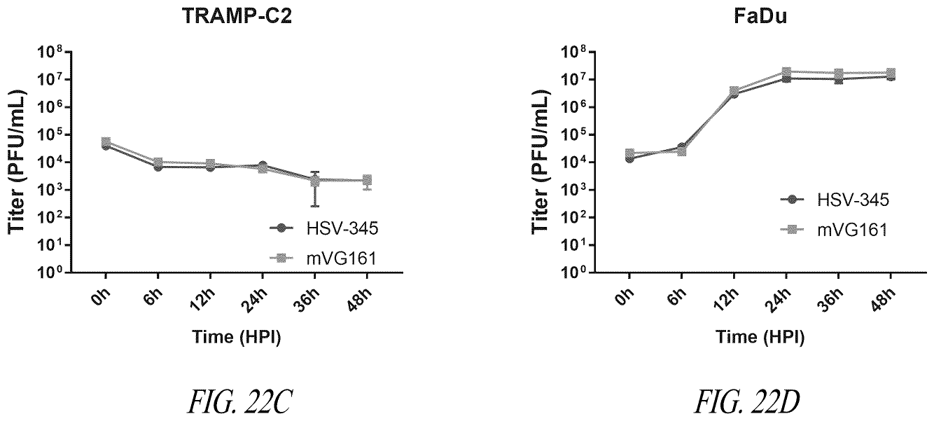

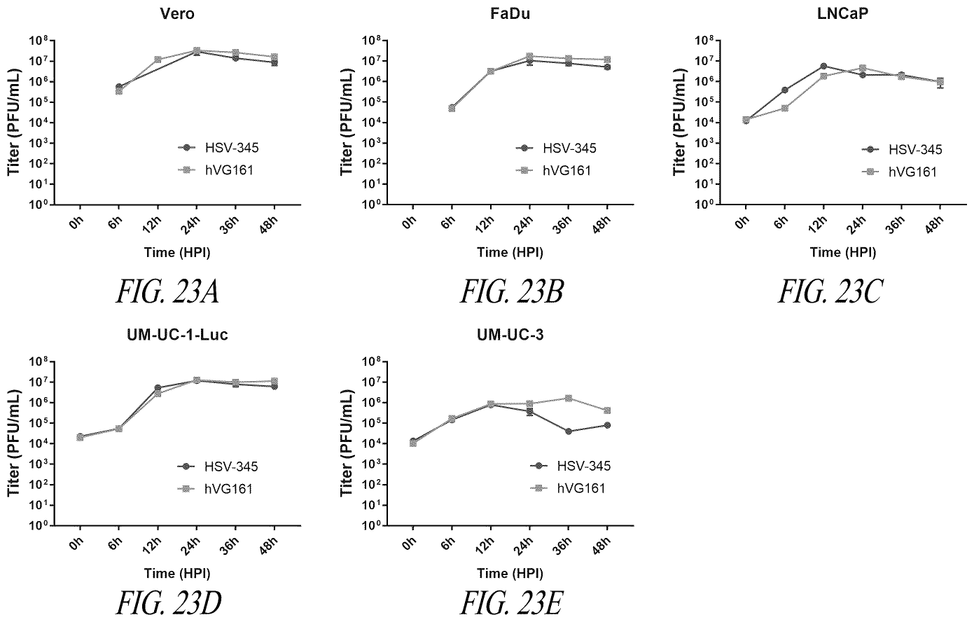

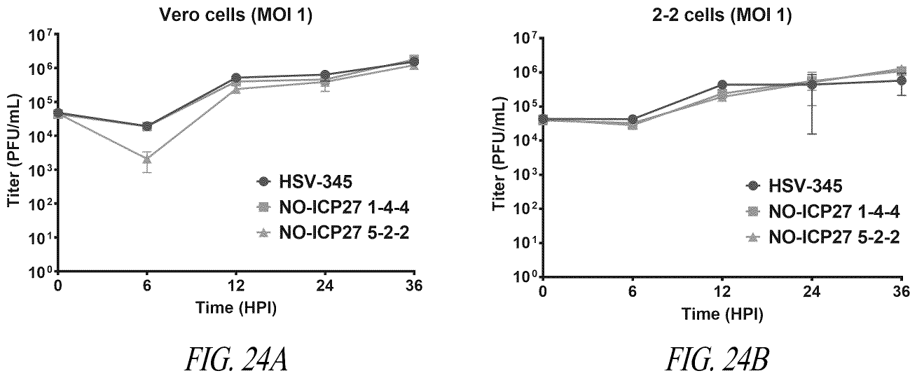

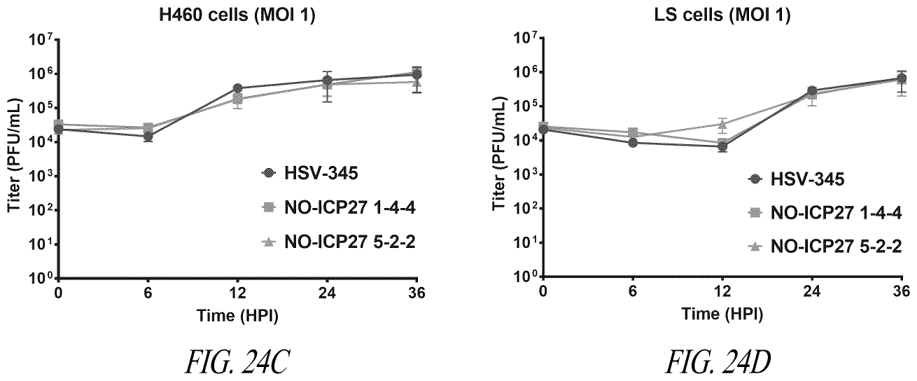

The growth curve and cytotoxicity data in FIGS. 21-24 show that hVG161 viruses replicate as well as the parental HSV-345 virus. These data also show that the viruses do not grow as well in mouse tumor cell lines compared to human cell lines, but HSV-1 is known to grow poorly in mouse cells.

The following are additional exemplary embodiments of the present disclosure:
1) An HSV vector comprising an NF-κB response element in a regulatory region of a viral gene that affects viral replication efficiency.
2) A HSV vector comprising an Oct-3/4-SOX2 response element in a regulatory region of viral gene that affects viral replication efficiency.
3) The vector of embodiments 1 or 2, wherein the viral gene encodes ICP4, ICP27, US11 or ICP8. Within further aspects of embodiments 1 and/or 2, the HSV vector is an HSV-1 vector.
4) The vector of any of embodiments 1 to 3, wherein the NF-κB response element comprises from 1-15 tandem sequences of GGGAATTTCC (SEQ ID NO: 1) or variants in Table 1.
5) The vector of embodiment 4, wherein the tandem sequences are identical or a mix of identical and different sequences or all different sequences.
6) The vector of embodiment 4, wherein the NF-κB response element has the sequence GGGAATTTCCGGGGACTTTCCGGGAATTTCCGGGGACTTTCCGGGAATTTCC (SEQ ID NO: 41).
7) The vector of embodiment 2, where the OCT-3/4-SOX2 complex response element comprises SEQ ID NO: 2 or a variant thereof, wherein the variant has at least 90% identical nucleotides.
8) The HSV vector of any one of embodiments 1-7, further comprising a sequence encoding a therapeutic substance for cancer treatment.
9) The HSV vector of embodiment 8, wherein the therapeutic substance is IL12, IL15, OX40L, PDL-1 blocker and/or a PD-1 blocker.
10) A method of treating cancer, comprising administering an HSV-1 vector according to any one of embodiments 1-9 to a patient in need.
11) A method of treating cancer stem cells and treatment-resistant cancer, comprising administering an HSV vector according to any one of embodiments 1-9 to a patient with a treatment resistant cancer or a cancer having cancer stem cells.
12) The method of embodiments 10-11, wherein the cancers are colon cancer, lung cancer, breast cancer, prostate cancer, brain cancer, or bladder cancer.

From the foregoing it will be appreciated that, although specific embodiments have been described herein for purposes of illustration, various modifications may be made without deviating from the spirit and scope of the invention. Accordingly, the invention is not limited except as by the appended claims

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

1-12. (canceled)

13. An HSV vector comprising an Oct-3/4-SOX2 response element in a regulatory region of viral gene that affects viral replication efficiency, and the viral gene encodes ICP4 or ICP27.

14. An HSV vector comprising an Oct-3/4-SOX2 response element in a regulatory region of a viral gene that affects viral replication efficiency, wherein the Oct-3/4-SOX2 response element is selected from:
CTACAGAGGTGCATATTAACAGAGCTTTTGTCCTGGAGA (SEQ ID NO: 2),
CTTTGTTATGCATCT (SEQ ID NO: 35), CATTGTGATGCATAT (SEQ ID NO: 36),
CATTGTAATGCAAAA (SEQ ID NO: 37), CATTGTTATGCTAGT (SEQ ID NO: 38),
CATTGTTATGCTAGT (SEQ ID NO: 39), and CTTTGTTTGGATGCTAAT (SEQ ID NO: 40), or a variant thereof, wherein the variant has at least 90% identical nucleotides.

15. The HSV vector of claim 13, wherein the viral gene encodes ICP4 or ICP27.

16. The HSV vector of claim 13 or 14, further comprising a sequence encoding a therapeutic substance for cancer treatment.

17. The HSV vector of claim 16, wherein the therapeutic substance is IL12, IL15, OX40L, PDL-1 blocker or a PD-1 blocker.

18. A method of treating cancer, comprising administering an HSV vector of claim 13 or 14 to a patient in need.

19. A method of treating cancer stem cells and treatment-resistant cancer, comprising administering an HSV vector of claim 13 or 14 to a patient with a treatment resistant cancer or a cancer having cancer stem cells.

20. The method of claim 19, wherein the cancers are colon cancer, lung cancer, breast cancer, prostate cancer, brain cancer, or bladder cancer.


SEQUENCE LISTING (41 sequences)

SEQ ID NO: 1 - DNA, 10 bp, Homo sapiens
  source 1..10: mol_type = other DNA
gggaatttcc

SEQ ID NO: 2 - DNA, 39 bp, Homo sapiens
  source 1..39: mol_type = genomic DNA
ctacagaggtgcatattaacagagcttttgtcctggaga

SEQ ID NO: 3 - DNA, 2894 bp, herpes simplex virus
  source 1..2894: mol_type = genomic DNA
atgctcgccgtccgttccctgcagcacctctcaaccgtcgtcttgataacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacctgacgccatgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggcgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcaatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgctgttccatcatcctctcgggcatcgcagtgcgattgtgttatatcgccgtggtggccggggtggtgctcgtggcgcttcactacgagcaggagatccagaggcgcctgtttgatgtatgacgtcactacagaggtgcatattaacagagcttttgtcctggagaaagcttcgcgttctttgaaagcagtcgagggggcgctagatgtgggcagggacgagctggcgcggcgtcgctgggtgcaccgcgaccacgggcagagccacgcggcgggaggactacaactcccggcacaccccgcgccgccccgcctctactcccagaaggccgcggggggtggaccgcctaagagggcgtgcgctcccgacatgccccgcggcgcgccattaaccgccagatttgaatcgcgggacccgttggcagaggtggcggcggcggcaaagggcgaattgatccgtcatggcgactgacattgatatgctaattgacctcggcctggacctctccgacagcgatctggacgaggacccccccgagccggcggagagccgccgcgacgacctggaatcggacagcagcggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcagcacgggggcaaggtggcccgcctccaacccccaccgaccaaagcccagcctgcccgcggcggacgccgtgggcgtcgcaggggtcggggtcgcggtggtcccggggctgccgatggtttgtcggacccccgccggcgtgcccccagaaccaatcgcaaccctgggggaccccgccccggggcggggtggacggacggccccggcgccccccatggcgaggcgtggcgcggcagtgagcagcccgacccacccggaggccagcggacacggggcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagctttggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggctgtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttag

SEQ ID NO: 4 - DNA, 3185 bp, herpes simplex virus
  source 1..3185: mol_type = genomic DNA
atgctcgccgtccgttccctgcagcacctctcaaccgtcgtcttgataacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacctgacgccatgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggcgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgctgttccatcatcctctcgggcatcgcagtgcgattgtgttatatcgccgtggtggccggggtggtgctcgtggcgcttcactacgagcaggagatccagaggcgcctgtttgatgtatgacgtcacatccaggccggcggaaaccgtaacggcatatgcaaattggaaactgtcctgtcttggggcccacccacccgacgcgtcatatgcaaatgaaaatcggtcccccgaggccacgtgtagcctggatcccaacgaccccgcccatgggtcccaattggccgtcccgttaccaagaccaacccagccagcatatccacccccgcccgggtccccgcggaagcggaacggggtatgtgatatgctaattaaatacgggaatttccggggactttccgggaatttccggggactttccgggaatttccctacagaggtgcatattaacagagcttttgtcctggagaatgccacgtacttatggtgtctgattggtccttgtctgtgccggaggtggggcgggggccccgcccggggggcggaacgaggaggggtttgggagagccggccccggcaccacgggtataaggacatccaccacccggccggtggtggtgtgcagccgtgttccaaccacggtcacgcttcggtgcctctccccgattcgggcccggtcgctcgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcagcggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcagcacgggggcaaggtggcccgcctccaacccccaccgaccaaagcccagcctgcccgcggcggacgccgtgggcgtcgcaggggtcggggtcgcggtggtcccggggctgccgatggtttgtcggacccccgccggcgtgcccccagaaccaatcgcaaccctgggggaccccgccccggggcggggtggacggacggccccggcgccccccatggcgaggcgtggcgcggcagtgagcagcccgacccacccggaggccagcggacacggggcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagctttggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggctgtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttag

SEQ ID NO: 5 - DNA, 3185 bp, herpes simplex virus
  source 1..3185: mol_type = genomic DNA
atgctcgccgtccgttccctgcagcacctctcaaccgtcgtcttgataacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacctgacgccatgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggcgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgctgttccatcatcctctcgggcatcgcagtgcgattgtgttatatcgccgtggtggccggggtggtgctcgtggcgcttcactacgagcaggagatccagaggcgcctgtttgatgtatgacgtcacatccaggccggcggaaaccgtaacggcatatgcaaattggaaactgtcctgtcttggggcccacccacccgacgcgtcatatgcaaatgaaaatcggtcccccgaggccacgtgtagcctggatcccaacgaccccgcccatgggtcccaattggccgtcccgttaccaagaccaacccagccagcatatccacccccgcccgggtccccgcggaagcggaacggtgtatgtgatatgctaattaaatacatgccacgtacttatggtgtctgattggtccttgtctgtgccggaggggaatttccggggactttccgggaatttccggggactttccgggaatttccctacagaggtgcatattaacagagcttttgtcctggagagtggggcgggggccccgcccggggggcggaacgaggaggggtttgggagagccggccccggcaccacgggtataaggacatccaccacccggccggtggtggtgtgcagccgtgttccaaccacggtcacgcttcggtgcctctccccgattcgggcccggtcgctcgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcagcggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcagcacgggggcaaggtggcccgcctccaacccccaccgaccaaagcccagcctgcccgcggcggacgccgtgggcgtcgcaggggtcggggtcgcggtggtcccggggctgccgatggtttgtcggacccccgccggcgtgcccccagaaccaatcgcaaccctgggggaccccgccccggggcggggtggacggacggccccggcgccccccatggcgaggcgtggcgcggcagtgagcagcccgacccacccggaggccagcggacacggggcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagctttggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggctgtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttag

SEQ ID NO: 6 - DNA, 1230 bp, herpes simplex virus
  source 1..1230: mol_type = genomic DNA
gtgacgattcccccaatggccgcgcgtcccaggggaggcaggcccaccgcggggcggccccgtccccggggaccaacccggcgcccccaaagaatatcattagcatgcacggcccggcccccgatttgggggcccaacccggtgtcccccaaagaaccccattagcatgcccctcccgccgacgcaacaggggcttggcctgcgtcggtgccccggggcttcccgccttcccgaagaaactcattaccatacccggaaccccaggggaccaatgcgggttcattgagcgacccgcgggccaatgcgcgaggggccgtgtgttccgccaaaaaagcaattagcataacccggaaccccaggggagtggttacgcgcggcgcgggaggcggggaataccggggttgcccattaagggccgcgggaattgccggaagcgggaagggcggccggggccgcccattagttacctgggactgtgcggttgggacggcgcccgtgggcccgggcggccgggggcggcgggggccgcgatggcggcggcggcgggccatggagacagagagcgtgccggggtggtagagtttgacaggcaagcatgtgcgtgcagaggcgagtagtgcttgcctgtctaactcgctagtctcggccgcggggggcccgggctgcccgccgccgccgctttaaagggccgcgcgcgacccccggggggtgtgttttggggggggcccgttttcggggtctggccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcccgcggccccgccccccacgcccgccgcgcgcgcgcacgccgcccggaccgccgcccgccttttttgcgcgcgcgcgcgcccgcggggggcccgggctgccacaggtgaaaccaacagagcacggcgcactccgcacgtcacacgtcacgtcatccaccacacctgcccaacaacacaactcacagcgacaactcaccgcgcaacaactcctgttcctcatccacacgtcaccgcgcacctcccgctcctccagacgtaccccgg

SEQ ID NO: 7 - DNA, 3185 bp, herpes simplex virus; identical to SEQ ID NO: 4
  source 1..3185: mol_type = genomic DNA

SEQ ID NO: 8 - DNA, 3670 bp, herpes simplex virus
  source 1..3670: mol_type = genomic DNA
atgtcgggcgtcgggggagagggagttccctctgcgcttgcgattctagcctcgtggggctggacgttcgacacgccaaaccacgagtcggggatatcgccagatacgactcccgcagattccattcggggtgccgctgtggcctcacctgaccaacctttacacgggggcccggaacgggaggccacagcgccgtctttctccccaacgcgcgcggatgacggcccgccctgtaccgacgggccctacgtgacgtttgataccctgtttatggtgtcgtcgatcgacgaattagggcgtcgccagctcacggacaccatccgcaaggacctgcggttgtcgctggccaagtttagcattgcgtgcaccaagacctcctcgttttcgggaaacgccccgcgccaccacagacgcggggcgttccagcgcggcacgcgggcgccgcgcagcaacaaaagcctccagatgtttgtgttgtgcaaacgcgcccacgccgctcgagtgcgagagcagcttcgggtcgttattcagtcccgcaagccgcgcaagtattacacgcgatcttcggacgggcggctctgccccgccgtccccgtgttcgtccacgagttcgtctcgtccgagccaatgcgcctccaccgagataacgtcatgctggcctcgggggccgagtaaccgcccccccgcgccaccctcactgcccgtcgcgcgtgtttgatgttaataaataacgcataaatttggctggttgtttgttgtctttaatggaccgcccgcagggggggtggcatttcagtgtcgggtgacgagcgcgatccggccgggagaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcatgtacaggatgcaactcctgtcttgcattgcactaagtcttgcacttgtcacgaattcgatatcgacagcccacccctctcctagcccaagatccgccggccagttcgccatggttagatctcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatgagtggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcctaggaccccaaaagtttgtctgcgtattccagggcggggctcagttgaatctcccgcagcacctctaccagcaggtccgcggtgggctggagaaactcggccgtcccggggcaggcggtcgtcgggggtggaggcgcggcgcccaccccgtgtgccgcgcctggcgtctcctctgggggcgacccgtaaatggttgcagtgatgtaaatggtgtccgcggtccagaccacggtcaaaatgccggccgtggcgctccgggcgctttcgccgcgcgaggagctgacccaggagtcgaacggatacgcgtacatatgggcgtcccacccgcgttcgagcttctggttgctgtcccggcctataaagcggtaggcacaaaattcggcgcgacagtcgataatcaccaacagcccaatgggggtgtgctggataacaacgcctccgcgcggcaggcggtcctggcgctcccggccccgtaccatgatcgcgcgggtgccgtactcaaaaacatgcaccacctgcgcggcgtcgggcagtgcgctggtcagcgaggccctggcgtggcataggctatacgcgatggtcgtctgtggattggacat

SEQ ID NO: 9 - DNA, 6903 bp, herpes simplex virus
  source 1..6903: mol_type = genomic DNA
ttagaggcgccgggagtggggtcgtcggcgtctgctttttgtggcggcgtcccgtcgcggggtggggtccgacgtggcgatgatgggcggcggcgtggtgaggggcttcggctgcaggcccgcttcatcgtccggcggcagaaccggggtccgtccagacgttccgttggtaggtcccaaatcctgtcgccctacacagcggcgggtgcgcgaatagtcaaagttcacacacccagccttcacaggtgtgtggctggcggcctgcttgcgactgtccagcgcctggcgtatctctttataaagggccaagcgcgtttctgtttcctgggtgttggcaggaaacgcggggtaactcaagtcctccaaaaaacccgccacaaataaaaaggggttaacccaatatgccttctgggcatgcctatcccacaagaccgtgtccaatccgggacagtgataacgcaaaaatataccgtctatcaaagcatagtttatagccgagggggtctcgtaacgccaatcaagatcgtcagacgggagcggcacacaaggcacctttaatatatcccccacctctcgagccacccgactccgaataacatattcggttgaagacaagcccccccgcacacacaaaaccccaacggcaataagcccgacccaacccaaaatccccatagcgcctagggtcggcacccacagaaacctacagtccccaagtgtttgcccagtaacacaaccacgacgtcgtgccacacaagccccgtatccccgttcccgcgcttttcgttggtttatatacacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccaccatgtgccatcagcagctggtcatctcatggttctccctggtgtttctggcctcacctctggtcgcaatctgggaactgaaaaaggatgtgtacgtggtggagctggactggtatcccgatgcccctggcgagatggtggtgctgacctgcgacacacccgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggtccacagacatcctgaaggatcagaaggagccaaagaacaagaccttcctgcggtgcgaggccaagaattatagcggccggttcacctgttggtggctgaccacaatctccaccgatctgacattttctgtgaagtctagcaggggatcctctgacccacagggagtgacatgcggagcagccaccctgagcgccgagagggtgcgcggcgataacaaggagtacgagtattccgtggagtgccaggaggactctgcctgtccagcagcagaggagtccctgcctatcgaagtgatggtggatgccgtgcacaagctgaagtacgagaattataccagctccttctttatccgggacatcatcaagcccgatccccctaagaacctgcagctgaagcctctgaagaatagcagacaggtggaggtgtcctgggagtaccctgacacctggagcacaccacactcctatttctctctgaccttttgcgtgcaggtgcagggcaagtccaagcgggagaagaaggacagagtgttcaccgataagacatctgccaccgtgatctgtagaaagaacgcctctatcagcgtgagggcccaggaccgctactattctagctcctggtccgagtgggcctctgtgccttgcagcggcggaggaggaggaggatctatgtgcccagcaaggagcctgctgctggtggccacactggtgctgctggatcacctgtccctggcaaggaatctgccagtggcaacccctgacccaggcatgttcccctgcctgcaccacagccagaacctgctgagggccgtgtccaatatgctgcagaaggcccgccagacactggagttttacccttgtaccagcgaggagatcgaccacgaggacatcacaaaggataagacctccacagtggaggcctgcctgccactggagctgaccaagaacgagtcctgtctgaacagccgggagacaagcttcatcaccaacggctcctgcctggcctctagaaagacaagctttatgatggccctgtgcctgtctagcatctacgaggacctgaagatgtatcaggtggagttcaagaccatgaacgccaagctgctgatggaccccaagaggcagatctttctggatcagaatatgctggccgtgatcgacgagctgatgcaggccctgaacttcaatagcgagacagtgcctcagaagtcctctctggaggagccagatttctacaagaccaagatcaagctgtgcatcctgctgcacgcctttcggatcagagccgtgacaatcgaccgcgtgatgtcctatctgaacgcctctggaggaggaggaagcggaggaggaggatccggcggcggcggctccggctctggcgccaccaacttctccctgctgaagcaggcaggcgacgtggaggagaatccaggacctatgcggatctctaagcctcacctgagaagcatctccatccagtgctacctgtgcctgctgctgaacagccactttctgacagaggccggcatccacgtgttcatcctgggctgttttagcgccggcctgccaaagaccgaggcaaactgggtgaatgtgatctctgacctgaagaagatcgaggatctgatccagagcatgcacatcgatgccacactgtataccgagtctgacgtgcaccctagctgcaaggtgaccgccatgaagtgtttcctgctggagctgcaggtcatcagcctggagtccggcgacgcaagcatccacgatacagtggagaacctgatcatcctggccaacaatagcctgagctccaacggcaatgtgaccgagtccggctgcaaggagtgtgaggagctggaggagaagaacatcaaggagttcctgcagagctttgtgcacatcgtgcagatgttcatcaatacatccggaggaggaggatcaggcggaggaggaagcggcggcggcggctctggcagcggcgccaccaacttttccctgctgaagcaggccggcgatgtggaagaaaatccaggaccaatggcaccaaggagagcaaggggatgcagaaccctgggcctgcctgccctgttattactgctgctgctgaggccaccagcaacaaggggaatcacctgtcctccacccatgagcgtggagcacgccgacatctgggtgaagtcctactctctgtattctagggagcggtacatctgcaacagcggctttaagaggaaggccggcacatctagcctgaccgagtgcgtgctgaacaaggccaccaatgtggcccactggaccacaccttccctgaagtgcatcagggatccagccctggtgcaccagcgccccgcacctccaagcacagtgaccacagcaggagtgacccctcagccagagagcctgtccccttctggcaaggagccagcagcatcctctccaagctccaacaatacagcagcaaccacagcagcaatcgtgccaggctcccagctgatgccaagcaagtccccctctaccggcaccacagagatctctagccacgagtcctctcacggcacaccaagccagaccacagccaagaattgggagctgaccgcaagcgcctcccaccagccacctggcgtgtacccacagggacactccgacaccacagtggccatctctaccagcacagtgctgctgtgcggcctgtctgccgtgagcctgctggcctgttatctgaagtccaggcagaccccaccactggcatctgtggagatggaggccatggaggccctgcccgtcacatgggggactagtagtagagacgaggatctggaaaactgtagtcaccatctgtaagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatccactagttctagagcggccgccgcacataaaggcccggcgcgaccgacgcccgcagacggcgccggccacgaacgacgggagcggctgcggagcacgcggaccgggagcgggactcgcagagggccgtcggagcggacggcgtcggcatcgcgacgccccggctcgggatcgggatcgcatcggaaagggacacgcggaaagacccacccaccccacccacgaaacacaggggacgcaccccgggggcctccgacgacagaaacccaccggtccgcctttgtgcacgggtaagcaccttgggtgggcggaggaggggggacgcgggggcggaggagggggctcacccgcgttcgtgccttcccgcaggaggaacgtcctcgtcgaggcgaccggcggcgaccgttgcgtggaccgcttcctgctcgtcggggcgaccggcggcgaccgttgcgtggaccgcttcctgctcgtcgggcgggggagcatgtcgtgggccctggaaatggcggacaccttcctggacaacatgcgggttgggcccaggacgtacgccgacgtacgcgatgagatcaataaaagggggcgtgaggaccgggaggcggccaaaaccgccgtgcacgacccggagcgtcctctgctgcgctctcccgggctgctgcccaaaatcgcccccaacgcatccttgggtgtggcacatcgaagaaccggcgggaccgtgaccgacagtccccgtaatccggtaacccgttgagtcccgggtacgaccatcgcccaagtttctgggcggagggtggttccccccgtggctctcgagatgagccagacccaacccccggccccagttgggccgggcgacccagatgtttacttaaaaggcgtgccgtccgccggcatgcaccccagaggtgttcacgcacctcgaggacacccgcgcatgatctccggacccccgcaacggggtgataatgatcaagcggcggggcaatgtggagattcgggtctactacgagtcggtgcggacactacgatctcgaagccatctgaagccgtccgaccgccaacaatccccaggacaccgcgtgttccccgggagccccgggttccgcgaccaccccgagaacctagggaacccagagtaccgcgagctcccagagaccccagggtaccgcgtgaccccagggatccacgacaaccccgggagccccggcctccccgggagccccggaccccacgcaccccccgcgaaccacgtacggctcgcgggtctgtatagcccgggcaagtatgcccccctggcgagcccagaccccttctccccacaagatggagcgtacgctcgggcccgcgtagggctccacaccgcggttcgcgtcccgcccaccggaagcccaacccacacgcacttgcggcatgacccgggcgatgagccaacctcggatgactcagggctctaccctctggacgcccgggcgcttgcgcacctggtgatgttgcccgcggaccaccgggccttctttcgaaccgtggtcgaggtgtctcgcatgtgcgctgcaaacgtgcgcgatcccccgcccccggctacaggggccatgttgggccgccacgcgcggctggtccacacccagtggctccgggccaaccaagagacgtcgcccctgtggccctggcggacggcggccattaactttatcaccaccatggccccccgcgtccaaacccaccgacacatgcacgacctgttgatggcctgtgctttctggtgctgtctgacacacgcatcgacgtgttcgtacgcggggctgtactcgacccactgcctgcatctgtttggtgcgtttgggtgtggggacccggccctaaccccacccctgtgctag

SEQ ID NO: 10 - DNA, 10 bp, Homo sapiens
  source 1..10: mol_type = genomic DNA
ggggatttcc

SEQ ID NO: 11 - DNA, 10 bp, Homo sapiens
  source 1..10: mol_type = genomic DNA
gggagattcc

SEQ ID NO: 12 - DNA, 10 bp, Homo sapiens
  source 1..10: mol_type = genomic DNA
gggaatctcc

SEQ ID NO: 13 - DNA, 10 bp, Homo sapiens
  source 1..10: mol_type = genomic DNA
gggagattcc

SEQ ID NO: 14 - DNA, 11 bp, Homo sapiens
  source 1..11: mol_type = genomic DNA
ggggattcccc

SEQ ID NO: 15 - DNA, 10 bp, Homo sapiens
  source 1..10: mol_type = genomic DNA
ggggaatccc

SEQ ID NO: 16 - DNA, 10 bp, Homo sapiens
  source 1..10: mol_type = genomic DNA
gggaatcccc

SEQ ID NO: 17 - DNA, 10 bp, Homo sapiens
  source 1..10: mol_type = genomic DNA
ggggaaggcc

SEQ ID NO: 18 - DNA, 10 bp, Homo sapiens
  source 1..10: mol_type = genomic DNA
gggaatttcc

SEQ ID NO: 19 - DNA, 10 bp, Homo sapiens
  source 1..10: mol_type = genomic DNA
gggaaattcc

SEQ ID NO: 20 - DNA, 10 bp, Homo sapiens
  source 1..10: mol_type = genomic DNA
gggaactacc

SEQ ID NO: 21 - DNA, 10 bp, Homo sapiens
  source 1..10: mol_type = genomic DNA
gggattttcc

SEQ ID NO: 22 - DNA, 10 bp, Homo sapiens
  source 1..10: mol_type = genomic DNA
gggatttcac

SEQ ID NO: 23 - DNA, 10 bp, Homo sapiens
  source 1..10: mol_type = genomic DNA
ggggctttcc

SEQ ID NO: 24 - DNA, 10 bp, Homo sapiens
  source 1..10: mol_type = genomic DNA
gggaaagccc

SEQ ID NO: 25 - DNA, 10 bp, Homo sapiens
  source 1..10: mol_type = genomic DNA
gggaattcac

SEQ ID NO: 26 - DNA, 10 bp, Homo sapiens
  source 1..10: mol_type = genomic DNA
gggggcttcc

SEQ ID NO: 27 - DNA, 10 bp, Homo sapiens
  source 1..10: mol_type = genomic DNA
ggggcttccc

SEQ ID NO: 28 - DNA, 10 bp, Homo sapiens
  source 1..10: mol_type = genomic DNA
gggaagcccc

SEQ ID NO: 29 - DNA, 10 bp, Homo sapiens
  source 1..10: mol_type = genomic DNA
ggggaagccc

SEQ ID NO: 30 - DNA, 10 bp, Homo sapiens
  source 1..10: mol_type = genomic DNA
gggaaatccc

SEQ ID NO: 31 - DNA, 10 bp, Homo sapiens
  source 1..10: mol_type = genomic DNA
gggactttcc

SEQ ID NO: 32 - DNA, 10 bp, Homo sapiens
  source 1..10: mol_type = genomic DNA
ggggctttcc

SEQ ID NO: 33 - DNA, 10 bp, Homo sapiens
  source 1..10: mol_type = genomic DNA
ggggaatccc

SEQ ID NO: 34 - DNA, 10 bp, Homo sapiens
  source 1..10: mol_type = genomic DNA
gggattcccc

SEQ ID NO: 35 - DNA, 15 bp, Homo sapiens
  source 1..15: mol_type = genomic DNA
ctttgttatgcatct

SEQ ID NO: 36 - DNA, 15 bp, Homo sapiens
  source 1..15: mol_type = genomic DNA
cattgtgatgcatat

SEQ ID NO: 37 - DNA, 15 bp, Homo sapiens
  source 1..15: mol_type = genomic DNA
cattgtaatgcaaaa

SEQ ID NO: 38 - DNA, 15 bp, Homo sapiens
  source 1..15: mol_type = genomic DNA
cattgttatgctagt

SEQ ID NO: 39 - DNA, 15 bp, Homo sapiens
  source 1..15: mol_type = genomic DNA
cattgttatgataaa

SEQ ID NO: 40 - DNA, 18 bp, Homo sapiens
  source 1..18: mol_type = genomic DNA
ctttgtttggatgctaat

SEQ ID NO: 41 - DNA, 52 bp, Homo sapiens
  source 1..52: mol_type = genomic DNA
gggaatttccggggactttccgggaatttccggggactttccgggaatttcc